SEQUENCE LISTING (12 sequences)

SEQ ID NO: 1 - protein, 574 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 2 - RNA, 1722 nt, synthetic construct
  source 1..1722: mol_type = other RNA
atggaactgctgatccacagatccagcgctattttcctgacactggccatcaatgccctgtacctgacaagcagccagaacatcacagaggaattctaccagagtacctgtagcgctgtgtctcggggatacctgagcgccctgcggaccggttggtacaccagcgtgatcacaatcgagctgagtaacatcaaggagacaaaatgcaatggcactgacaccaaggtgaaacttatcaaacaggagctggataagtacaagaatgccgtgacagaactgcagctgctgatgcagaatacccccgccgtgaacaacagagccagacgggaagcccctcaatacatgaactacacaatcaacaccacaaagaacctgaacgtgtctatctccaagaagcgcaagcggagattcctgggcttcctgttgggagtgggctctgccatcgccagcggcatcgcagtgtgcaaggtgctgcacctggaaggagaagtgaacaagatcaagaatgccctgcagctgaccaacaaggccgtggtctctctgagcaatggcgtgagcgtgctgacctttagagtgctggacctgaagaactacatcaacaaccagctgctgcctatgctgaacagacagtcttgtagaatcagcaacatcgaaaccgtgatcgagttccagcagaagaactccaggctgctggaaatcacaagagagtttagcgtgaacgccggcgtcaccacccccctgagcacctacatgctgaccaacagcgagctgctgagcctgattaacgacatgcctatcaccaatgaccagaaaaagctaatgagcagcaatgtgcaaatcgtgcggcagcagtcctacagcatcatgtgcatcatcaaggaagaggtcctggcctacgtggtacaactgcctatctacggcgtgatcgacaccccttgttggaagctccataccagccctctgtgcacaacaaacatcaaagaaggcagcaatatctgcctgacaagaacagatagaggctggtattgtgacaacgctggctccgtctccttcttccctcaggccgacacctgcaaggtgcagagcaaccgcgtgttctgcgataccatgaacagcttaacactgcccagcgaggtgtctctctgtaacaccgacatcttcaactctaaatacgactgcaagatcatgacctctaagactgatatcagcagcagcgtgattacctccctgggcgccatcgtgtcttgctacggcaagacaaagtgcaccgccagcaacaagaaccggggcatcatcaagaccttcagcaacggctgcgactacgtgagcaacaagggcgtggacaccgttagcgtgggcaacacactgtattacgtaaataaacttgagggcaagaatctgtacgtgaagggcgagcctatcatcaactactacgacccactggtgtttccatctgatgagttcgacgcctccatctcccaggtgaacgagaagatcaaccagagcctggcttttatcagaagaagcgatgagctgctgcacaacgtgaataccggaaaaagcaccaccaacatcatgatcaccgccatcattatcgttatcatcgtggtgctgctgagtctgatcgccattggactgctgctgtattgcaaagccaagaacacccccgtgaccctgagcaaggaccagctgagcggcatcaacaacatagctttctccaag

SEQ ID NO: 3 - RNA, 2015 nt, synthetic construct
  source 1..2015: mol_type = other RNA
aggccggcactcttctggtccccacagactcagagagaacccgccgccaccatggaactgctgatccacagatccagcgctattttcctgacactggccatcaatgccctgtacctgacaagcagccagaacatcacagaggaattctaccagagtacctgtagcgctgtgtctcggggatacctgagcgccctgcggaccggttggtacaccagcgtgatcacaatcgagctgagtaacatcaaggagacaaaatgcaatggcactgacaccaaggtgaaacttatcaaacaggagctggataagtacaagaatgccgtgacagaactgcagctgctgatgcagaatacccccgccgtgaacaacagagccagacgggaagcccctcaatacatgaactacacaatcaacaccacaaagaacctgaacgtgtctatctccaagaagcgcaagcggagattcctgggcttcctgttgggagtgggctctgccatcgccagcggcatcgcagtgtgcaaggtgctgcacctggaaggagaagtgaacaagatcaagaatgccctgcagctgaccaacaaggccgtggtctctctgagcaatggcgtgagcgtgctgacctttagagtgctggacctgaagaactacatcaacaaccagctgctgcctatgctgaacagacagtcttgtagaatcagcaacatcgaaaccgtgatcgagttccagcagaagaactccaggctgctggaaatcacaagagagtttagcgtgaacgccggcgtcaccacccccctgagcacctacatgctgaccaacagcgagctgctgagcctgattaacgacatgcctatcaccaatgaccagaaaaagctaatgagcagcaatgtgcaaatcgtgcggcagcagtcctacagcatcatgtgcatcatcaaggaagaggtcctggcctacgtggtacaactgcctatctacggcgtgatcgacaccccttgttggaagctccataccagccctctgtgcacaacaaacatcaaagaaggcagcaatatctgcctgacaagaacagatagaggctggtattgtgacaacgctggctccgtctccttcttccctcaggccgacacctgcaaggtgcagagcaaccgcgtgttctgcgataccatgaacagcttaacactgcccagcgaggtgtctctctgtaacaccgacatcttcaactctaaatacgactgcaagatcatgacctctaagactgatatcagcagcagcgtgattacctccctgggcgccatcgtgtcttgctacggcaagacaaagtgcaccgccagcaacaagaaccggggcatcatcaagaccttcagcaacggctgcgactacgtgagcaacaagggcgtggacaccgttagcgtgggcaacacactgtattacgtaaataaacttgagggcaagaatctgtacgtgaagggcgagcctatcatcaactactacgacccactggtgtttccatctgatgagttcgacgcctccatctcccaggtgaacgagaagatcaaccagagcctggcttttatcagaagaagcgatgagctgctgcacaacgtgaataccggaaaaagcaccaccaacatcatgatcaccgccatcattatcgttatcatcgtggtgctgctgagtctgatcgccattggactgctgctgtattgcaaagccaagaacacccccgtgaccctgagcaaggaccagctgagcggcatcaacaacatagctttctccaagtgactcgagtaagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 4757 bp, synthetic construct
  source 1..4757: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagattctagattaatacgactcactataaggccggcactcttctggtccccacagactcagagagaacccgccgccaccatggaactgctgatccacagatccagcgctattttcctgacactggccatcaatgccctgtacctgacaagcagccagaacatcacagaggaattctaccagagtacctgtagcgctgtgtctcggggatacctgagcgccctgcggaccggttggtacaccagcgtgatcacaatcgagctgagtaacatcaaggagacaaaatgcaatggcactgacaccaaggtgaaacttatcaaacaggagctggataagtacaagaatgccgtgacagaactgcagctgctgatgcagaatacccccgccgtgaacaacagagccagacgggaagcccctcaatacatgaactacacaatcaacaccacaaagaacctgaacgtgtctatctccaagaagcgcaagcggagattcctgggcttcctgttgggagtgggctctgccatcgccagcggcatcgcagtgtgcaaggtgctgcacctggaaggagaagtgaacaagatcaagaatgccctgcagctgaccaacaaggccgtggtctctctgagcaatggcgtgagcgtgctgacctttagagtgctggacctgaagaactacatcaacaaccagctgctgcctatgctgaacagacagtcttgtagaatcagcaacatcgaaaccgtgatcgagttccagcagaagaactccaggctgctggaaatcacaagagagtttagcgtgaacgccggcgtcaccacccccctgagcacctacatgctgaccaacagcgagctgctgagcctgattaacgacatgcctatcaccaatgaccagaaaaagctaatgagcagcaatgtgcaaatcgtgcggcagcagtcctacagcatcatgtgcatcatcaaggaagaggtcctggcctacgtggtacaactgcctatctacggcgtgatcgacaccccttgttggaagctccataccagccctctgtgcacaacaaacatcaaagaaggcagcaatatctgcctgacaagaacagatagaggctggtattgtgacaacgctggctccgtctccttcttccctcaggccgacacctgcaaggtgcagagcaaccgcgtgttctgcgataccatgaacagcttaacactgcccagcgaggtgtctctctgtaacaccgacatcttcaactctaaatacgactgcaagatcatgacctctaagactgatatcagcagcagcgtgattacctccctgggcgccatcgtgtcttgctacggcaagacaaagtgcaccgccagcaacaagaaccggggcatcatcaagaccttcagcaacggctgcgactacgtgagcaacaagggcgtggacaccgttagcgtgggcaacacactgtattacgtaaataaacttgagggcaagaatctgtacgtgaagggcgagcctatcatcaactactacgacccactggtgtttccatctgatgagttcgacgcctccatctcccaggtgaacgagaagatcaaccagagcctggcttttatcagaagaagcgatgagctgctgcacaacgtgaataccggaaaaagcaccaccaacatcatgatcaccgccatcattatcgttatcatcgtggtgctgctgagtctgatcgccattggactgctgctgtattgcaaagccaagaacacccccgtgaccctgagcaaggaccagctgagcggcatcaacaacatagctttctccaagtgactcgagtaagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 6 - RNA, 1722 nt, synthetic construct
  source 1..1722: mol_type = other RNA
atggaactgctgatcctgaaggccaacgctatcacaaccatcctcaccgccgtgacattctgcttcgccagcggccagaacatcaccgaagagttctaccagagcacctgctccgctgtgtctaaagggtacctgtccgccctgagaaccggctggtataccagcgtgattacgattgagctgagcaacatcaagaagaacaagtgcaacggaacagacgccaaggtgaagctgatcaagcaggagctggataagtataagaacgccgtgaccgagttgcagctgctcatgcagtctacacaggccacaaacaatcgggccagaagagagctgcctagattcatgaactacaccctgaacaacgccaagaagacaaatgtgaccctgagcaagaaaagaaagcggagattcctgggcttcctgctcggcgttggctctgccatcgccagcggagtcgccgtgtgcaaagtgctgcacctggaaggagaagtgaacaagatcaagagcgccctgctgtctacgaacaaagccgtggtgagcctgtccaacggcgtgagcgtgctgaccttcaaggtgctggacctgaagaactacatcgataagcaactgctgccaatcctgaacaagcagtcttgtagcatcagcaatatcgagacagtgatcgagttccagcagaagaacaaccggctgctggaaatcacacgggaatttagcgtcaacgccggtgtgaccacccctgtgtctacctacatgctgaccaatagcgagctgctgagcctgatcaatgacatgcctattaccaacgaccaaaagaaactgatgagcaacaatgtacaaatcgttagacagcagagctactccatcatgtgcatcatcaaggaggaagtgctggcctacgtcgtgcaactgcctctgtacggcgtgatcgacaccccttgttggaagctgcacacgagccctctgtgcacaactaatacaaaggaaggcagcaacatctgcctgaccagaacagatagaggctggtactgcgacaacgctggatctgtcagctttttccctcaggctgaaacctgtaaagtgcagtccaaccgcgtgttttgcgatacaatgaactctctgacactgcctagcgaggtgaacctgtgtaatgtggacatcttcaaccccaagtacgattgcaagatcatgacaagcaagaccgatgtgtctagcagcgtgattaccagcctgggcgccatcgtgtcatgctacggcaagaccaagtgcaccgccagcaacaaaaatcggggcatcatcaaaaccttcagcaacggctgcgactacgtgagcaacaaaggagtggacaccgtctccgtgggcaacaccctgtactacgtgaacaagcaggagggcaagtctctgtacgtgaagggcgagccaatcattaacttctacgaccccctggtgttcccctctgatgagttcgacgcctccatcagccaggtgaacgagaagatcaaccagagcctggccttcatcagaaagagcgacgagctgctgcacaacgtcaatgccggcaaaagcaccaccaacatcatgatcaccaccatcatcatcgtgatcattgtgatcctgctgagtctgatcgctgtgggcctgctgctgtattgtaaagctaggtccacccccgtgacacttagcaaggaccagctgagcggcatcaacaacatcgcatttagcaac

SEQ ID NO: 7 - skipped

SEQ ID NO: 8 - DNA, 4620 bp, synthetic construct
  source 1..4620: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatggaactgctgatcctgaaggccaacgctatcacaaccatcctcaccgccgtgacattctgcttcgccagcggccagaacatcaccgaagagttctaccagagcacctgctccgctgtgtctaaagggtacctgtccgccctgagaaccggctggtataccagcgtgattacgattgagctgagcaacatcaagaagaacaagtgcaacggaacagacgccaaggtgaagctgatcaagcaggagctggataagtataagaacgccgtgaccgagttgcagctgctcatgcagtctacacaggccacaaacaatcgggccagaagagagctgcctagattcatgaactacaccctgaacaacgccaagaagacaaatgtgaccctgagcaagaaaagaaagcggagattcctgggcttcctgctcggcgttggctctgccatcgccagcggagtcgccgtgtgcaaagtgctgcacctggaaggagaagtgaacaagatcaagagcgccctgctgtctacgaacaaagccgtggtgagcctgtccaacggcgtgagcgtgctgaccttcaaggtgctggacctgaagaactacatcgataagcaactgctgccaatcctgaacaagcagtcttgtagcatcagcaatatcgagacagtgatcgagttccagcagaagaacaaccggctgctggaaatcacacgggaatttagcgtcaacgccggtgtgaccacccctgtgtctacctacatgctgaccaatagcgagctgctgagcctgatcaatgacatgcctattaccaacgaccaaaagaaactgatgagcaacaatgtacaaatcgttagacagcagagctactccatcatgtgcatcatcaaggaggaagtgctggcctacgtcgtgcaactgcctctgtacggcgtgatcgacaccccttgttggaagctgcacacgagccctctgtgcacaactaatacaaaggaaggcagcaacatctgcctgaccagaacagatagaggctggtactgcgacaacgctggatctgtcagctttttccctcaggctgaaacctgtaaagtgcagtccaaccgcgtgttttgcgatacaatgaactctctgacactgcctagcgaggtgaacctgtgtaatgtggacatcttcaaccccaagtacgattgcaagatcatgacaagcaagaccgatgtgtctagcagcgtgattaccagcctgggcgccatcgtgtcatgctacggcaagaccaagtgcaccgccagcaacaaaaatcggggcatcatcaaaaccttcagcaacggctgcgactacgtgagcaacaaaggagtggacaccgtctccgtgggcaacaccctgtactacgtgaacaagcaggagggcaagtctctgtacgtgaagggcgagccaatcattaacttctacgaccccctggtgttcccctctgatgagttcgacgcctccatcagccaggtgaacgagaagatcaaccagagcctggccttcatcagaaagagcgacgagctgctgcacaacgtcaatgccggcaaaagcaccaccaacatcatgatcaccaccatcatcatcgtgatcattgtgatcctgctgagtctgatcgctgtgggcctgctgctgtattgtaaagctaggtccacccccgtgacacttagcaaggaccagctgagcggcatcaacaacatcgcatttagcaactgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagggcccgtttaaacatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 9 - protein, 539 aa, synthetic construct
MSWKVVIIFSLLITPQHGLKESYLEESCSTITEGYLSVLRTGWYTNVFTLEVGDVENLTCADGPSLIKTELDLTKSALRELKTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNTGFIIVIILIAVLGSSMILVSICIIIKKTKKPTGAPPELSGVTNNGFIPHS

SEQ ID NO: 10 - RNA, 1617 nt, synthetic construct
  source 1..1617: mol_type = other RNA
atgagctggaaggtggtgatcatcttcagcctgctgatcacccctcagcacggcctgaaagagagctacctggaagaaagctgctctaccatcacagagggctacctgagtgtgctgcggaccggctggtacacaaacgtgtttaccctggaagtgggcgacgtggaaaacctgacatgcgccgatggccccagcctgattaagacagagctggacctgacaaagagcgccctgagagaactgaagacagtcagcgctgatcaactggccagggaggagcagatcgagaaccccagacagtccagattcgtgctcggtgccatcgccctgggcgtggcgacagctgccgctgttaccgctggcgtggctatcgccaagaccatcagactggaatctgaggtgaccgccatcaagaatgccctgaagaagaccaatgaggccgtgtctacactgggaaatggcgtgcgggtgctggctacagccgtccgggaactgaaagacttcgtgtccaagaacttgaccagagccatcaacaagaacaagtgcgatatcgacgacctgaagatggccgtgagcttcagccagttcaaccggcggttcctgaacgttgtgcggcagtttagcgacaacgccggaatcaccccagccatcagcctggacctgatgaccgacgccgagctggccagagccgtgagcaacatgcctacaagcgccggccagatcaagctgatgctggagaatcgggccatggtgagaagaaagggctttggcatcctgatcggcgtgtacggcagcagcgtgatctacatggtgcagctgcctatcttcggcgtgatcgataccccttgctggatcgtgaaggccgcccctagctgtagcgagaaaaagggaaactacgcctgtctgctgagagaagatcagggctggtattgccagaacgccggcagcaccgtgtactaccccaacgagaaggactgcgagacaagaggcgatcacgtgttctgcgacaccgccgccggcatcaacgttgctgagcaaagcaaggaatgcaacatcaacatcagcaccaccaactacccttgtaaagtgagcaccggaagacaccccatctccatggtcgcactctcccctctgggcgccctggtcgcctgctacaagggcgtgtcttgtagcatcggaagcaaccgcgttggcatcatcaagcagctgaacaaaggatgttcttatatcaccaaccaggatgccgacaccgtgacaatcgacaataccgtctaccagctgagcaaggtggaaggcgagcagcacgtgattaagggcagacctgtgtcctctagcttcgaccccgtgaagttccccgaggaccagttcaatgtggccctggatcaagtgtttgagaacatcgagaacagccaggccctggtggaccagagcaatagaatcctgtcctccgctgagaaaggcaacaccggcttcatcatcgtgatcatcctgatcgccgtgctgggctctagcatgatcctggtgtctatctgcattattatcaagaaaaccaagaagcctaccggcgctccacctgagctgagcggagtgaccaacaacggcttcatccctcattct

SEQ ID NO: 11 - RNA, 1910 nt, synthetic construct
  source 1..1910: mol_type = other RNA
aggccggcactcttctggtccccacagactcagagagaacccgccgccaccatgagctggaaggtggtgatcatcttcagcctgctgatcacccctcagcacggcctgaaagagagctacctggaagaaagctgctctaccatcacagagggctacctgagtgtgctgcggaccggctggtacacaaacgtgtttaccctggaagtgggcgacgtggaaaacctgacatgcgccgatggccccagcctgattaagacagagctggacctgacaaagagcgccctgagagaactgaagacagtcagcgctgatcaactggccagggaggagcagatcgagaaccccagacagtccagattcgtgctcggtgccatcgccctgggcgtggcgacagctgccgctgttaccgctggcgtggctatcgccaagaccatcagactggaatctgaggtgaccgccatcaagaatgccctgaagaagaccaatgaggccgtgtctacactgggaaatggcgtgcgggtgctggctacagccgtccgggaactgaaagacttcgtgtccaagaacttgaccagagccatcaacaagaacaagtgcgatatcgacgacctgaagatggccgtgagcttcagccagttcaaccggcggttcctgaacgttgtgcggcagtttagcgacaacgccggaatcaccccagccatcagcctggacctgatgaccgacgccgagctggccagagccgtgagcaacatgcctacaagcgccggccagatcaagctgatgctggagaatcgggccatggtgagaagaaagggctttggcatcctgatcggcgtgtacggcagcagcgtgatctacatggtgcagctgcctatcttcggcgtgatcgataccccttgctggatcgtgaaggccgcccctagctgtagcgagaaaaagggaaactacgcctgtctgctgagagaagatcagggctggtattgccagaacgccggcagcaccgtgtactaccccaacgagaaggactgcgagacaagaggcgatcacgtgttctgcgacaccgccgccggcatcaacgttgctgagcaaagcaaggaatgcaacatcaacatcagcaccaccaactacccttgtaaagtgagcaccggaagacaccccatctccatggtcgcactctcccctctgggcgccctggtcgcctgctacaagggcgtgtcttgtagcatcggaagcaaccgcgttggcatcatcaagcagctgaacaaaggatgttcttatatcaccaaccaggatgccgacaccgtgacaatcgacaataccgtctaccagctgagcaaggtggaaggcgagcagcacgtgattaagggcagacctgtgtcctctagcttcgaccccgtgaagttccccgaggaccagttcaatgtggccctggatcaagtgtttgagaacatcgagaacagccaggccctggtggaccagagcaatagaatcctgtcctccgctgagaaaggcaacaccggcttcatcatcgtgatcatcctgatcgccgtgctgggctctagcatgatcctggtgtctatctgcattattatcaagaaaaccaagaagcctaccggcgctccacctgagctgagcggagtgaccaacaacggcttcatccctcattcttgactcgagtaagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 4514 bp, synthetic construct
  source 1..4514: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgagctggaaggtggtgatcatcttcagcctgctgatcacccctcagcacggcctgaaagagagctacctggaagaaagctgctctaccatcacagagggctacctgagtgtgctgcggaccggctggtacacaaacgtgtttaccctggaagtgggcgacgtggaaaacctgacatgcgccgatggccccagcctgattaagacagagctggacctgacaaagagcgccctgagagaactgaagacagtcagcgctgatcaactggccagggaggagcagatcgagaaccccagacagtccagattcgtgctcggtgccatcgccctgggcgtggcgacagctgccgctgttaccgctggcgtggctatcgccaagaccatcagactggaatctgaggtgaccgccatcaagaatgccctgaagaagaccaatgaggccgtgtctacactgggaaatggcgtgcgggtgctggctacagccgtccgggaactgaaagacttcgtgtccaagaacttgaccagagccatcaacaagaacaagtgcgatatcgacgacctgaagatggccgtgagcttcagccagttcaaccggcggttcctgaacgttgtgcggcagtttagcgacaacgccggaatcaccccagccatcagcctggacctgatgaccgacgccgagctggccagagccgtgagcaacatgcctacaagcgccggccagatcaagctgatgctggagaatcgggccatggtgagaagaaagggctttggcatcctgatcggcgtgtacggcagcagcgtgatctacatggtgcagctgcctatcttcggcgtgatcgataccccttgctggatcgtgaaggccgcccctagctgtagcgagaaaaagggaaactacgcctgtctgctgagagaagatcagggctggtattgccagaacgccggcagcaccgtgtactaccccaacgagaaggactgcgagacaagaggcgatcacgtgttctgcgacaccgccgccggcatcaacgttgctgagcaaagcaaggaatgcaacatcaacatcagcaccaccaactacccttgtaaagtgagcaccggaagacaccccatctccatggtcgcactctcccctctgggcgccctggtcgcctgctacaagggcgtgtcttgtagcatcggaagcaaccgcgttggcatcatcaagcagctgaacaaaggatgttcttatatcaccaaccaggatgccgacaccgtgacaatcgacaataccgtctaccagctgagcaaggtggaaggcgagcagcacgtgattaagggcagacctgtgtcctctagcttcgaccccgtgaagttccccgaggaccagttcaatgtggccctggatcaagtgtttgagaacatcgagaacagccaggccctggtggaccagagcaatagaatcctgtcctccgctgagaaaggcaacaccggcttcatcatcgtgatcatcctgatcgccgtgctgggctctagcatgatcctggtgtctatctgcattattatcaagaaaaccaagaagcctaccggcgctccacctgagctgagcggagtgaccaacaacggcttcatccctcattcttgactcgagtaagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

(19) United States
(12) Patent Application Publication
(10) Pub. No.: US 2024/0261387 A1
(43) Pub. Date: Aug. 8, 2024

(54) RESPIRATORY SYNCYTIAL VIRUS MRNA VACCINE

(71) Applicant: Vernagen, LLC, Tucker, GA (US)

(72) Inventors: Baek KIM, Atlanta, GA (US); Jack YOON, Suwanee, GA (US); David PAK, Suwanee, GA (US)

(73) Assignee: Vernagen, LLC, Tucker, GA (US)

(21) Appl. No.: 18/394,486

(22) Filed: Dec. 22, 2023

Related U.S. Application Data
(60) Provisional application No. 63/483,099, filed on Feb. 3, 2023.

(51) Int. Cl. A61K 39/155; A61K 39/00; A61K 39/295; A61P 37/04

(52) U.S. Cl. CPC A61K 39/155; A61K 39/295; A61P 37/04; A61K 2039/53; A61K 2039/70; C12N 2760/18322; C12N 2760/18334; C12N 2760/18522; C12N 2760/18534

(57) ABSTRACT

Provided herein are a respiratory syncytial virus (RSV) vaccine composition including a messenger ribonucleic acid (mRNA) including an open reading frame (ORF) encoding RSV mutant F B strain protein, and optionally a mRNA including an ORF encoding RSV mutant F A strain protein, and a method of inducing immune response against RSV by administering an effective amount of the RSV vaccine composition to a subject in need thereof. Provided herein are also a respiratory syncytial virus (RSV) and human metapneumovirus virus (hMPV) vaccine composition including a mRNA including an ORF encoding RSV mutant F A strain protein, a mRNA including an ORF encoding RSV mutant F B strain protein, and a mRNA including an ORF encoding hMPV F protein, and a method of inducing immune response against RSV and hMPV by administering an effective amount of the RSV and hMPV vaccine composition to a subject in need thereof.

### CROSS REFERENCE TO RELATED APPLICATIONS

This application claims priority to U.S. Provisional Application No. 63/483,099 filed Feb. 3, 2023, the entire disclosure of which is incorporated herein by reference.

### INCORPORATION BY REFERENCE OF SEQUENCE LISTING

The content of the electronically submitted sequence listing, file name: A294002_substitute sequence listing as filed; size: 37,950 bytes; and date of creation: May 14, 2024, filed herewith, is incorporated herein by reference in its entirety.

### FIELD

Provided herein are a respiratory syncytial virus (RSV) vaccine composition including a messenger ribonucleic acid (mRNA) including an open reading frame (ORF) encoding RSV mutant F B strain protein, and optionally a mRNA including an ORF encoding RSV mutant F A strain protein, and a method of inducing immune response against RSV by administering an effective amount of the RSV vaccine composition to a subject in need thereof.

Provided herein are also a respiratory syncytial virus (RSV) and human metapneumovirus virus (hMPV) vaccine composition including a mRNA including an ORF encoding RSV mutant F A strain protein, a mRNA including an ORF encoding RSV mutant F B strain protein, and a mRNA including an ORF encoding hMPV F protein, and a method of inducing immune response against RSV and hMPV by administering an effective amount of the RSV and hMPV vaccine composition to a subject in need thereof.

### BACKGROUND

Respiratory syncytial virus (RSV) is a common respiratory virus that infects the nose, throat, lungs, and breathing passages. At present, there is no approved RSV vaccine, although the development of a vaccine would be one of the best strategies for preventing RSV. There has been a need for RSV vaccine.

Human metapneumovirus (hMPV) is a similar but separate virus. The seasonal epidemiology of hMPV is similar to that of RSV. At present, there is no approved RSV and hMPV vaccine, although the development of a vaccine would be one of the best strategies for preventing RSV and hMPV. There has been a need for RSV and hMPV vaccine.

### SUMMARY

The present disclosure provides a respiratory syncytial virus (RSV) vaccine composition comprising a messenger ribonucleic acid (mRNA) comprising an open reading frame (ORF) encoding RSV mutant F B strain protein having an amino acid sequence of SEQ ID NO: 1. In one embodiment, the ORF encoding RSV mutant F B strain protein has a nucleotide sequence of SEQ ID NO: 2. In one embodiment, the mRNA comprising the ORF encoding RSV mutant F B strain protein further comprises a 5′ untranslated region (UTR), a 3′ UTR, and a poly (A) tail so as to have the structure of 5′UTR-ORF encoding RSV mutant F B strain protein-3′UTR-poly (A) tail, and the ORF encoding RSV mutant F B strain protein has a nucleotide sequence of SEQ ID NO: 2. In one embodiment, the poly (A) tail has a length of 50-250 nucleotides. In one embodiment, the mRNA having the structure of 5′UTR-ORF encoding RSV mutant F B strain protein-3′UTR-poly (A) tail has a nucleotide sequence of SEQ ID NO: 3. In another embodiment, the mRNA having the structure of 5′UTR-ORF encoding RSV mutant F B strain protein-3′UTR-poly (A) tail has a nucleotide sequence having at least 80% identity to SEQ ID NO: 3.

The present disclosure also provides a RSV vaccine composition comprising a mRNA comprising an ORF encoding RSV mutant F B strain protein having an amino acid sequence of SEQ ID NO: 1, and a mRNA comprising an ORF encoding RSV mutant F A strain protein having an amino acid sequence of SEQ ID NO: 5. In one embodiment, the ORF encoding RSV mutant F A strain protein has a nucleotide sequence of SEQ ID NO: 6. In another embodiment, the mRNA comprising the ORF encoding RSV mutant F A strain protein further comprises a 5′ untranslated region (UTR), a 3′ UTR, and a poly (A) tail so as to have the structure of 5′UTR-ORF encoding RSV mutant F A strain protein-3′UTR-poly (A) tail, and the ORF encoding RSV mutant F A strain protein has a nucleotide sequence of SEQ ID NO: 6. In one embodiment, the poly (A) tail has a length of 50-250 nucleotides. In one embodiment, the mRNA having the structure of 5′UTR-ORF encoding RSV mutant F A strain protein-3′UTR-poly (A) tail has a nucleotide sequence of SEQ ID NO: 7. In another embodiment, the mRNA having the structure of 5′UTR-ORF encoding RSV mutant F A strain protein-3′UTR-poly (A) tail has a nucleotide sequence having at least 80% identity to SEQ ID NO: 7.

In one embodiment, the RSV vaccine composition of the present disclosure further comprises a pharmaceutically acceptable carrier. In one embodiment, the pharmaceutically acceptable carrier is a lipid nanoparticle encapsulating the mRNA therein.

The present disclosure also provides a method of inducing immune response against respiratory syncytial virus (RSV) comprising administering an effective amount of the RSV vaccine composition of the present disclosure to a subject in need thereof.

In addition, the present disclosure provides a respiratory syncytial virus (RSV) and human metapneumovirus virus (hMPV) vaccine composition comprising a messenger ribonucleic acid (mRNA) comprising an open reading frame (ORF) encoding RSV mutant F A strain protein having an amino acid sequence of SEQ ID NO: 5, a mRNA comprising an ORF encoding RSV mutant F B strain protein having an amino acid sequence of SEQ ID NO: 1, and a mRNA comprising an ORF encoding hMPV F protein having an amino acid sequence of SEQ ID NO: 9. In one embodiment, the ORF encoding RSV mutant F A strain protein has a nucleotide sequence of SEQ ID NO: 6, the ORF encoding RSV mutant F B strain protein has a nucleotide sequence of SEQ ID NO: 2, and the ORF encoding hMPV F protein has a nucleotide sequence of SEQ ID NO: 10. In another embodiment, the mRNA comprising the ORF encoding RSV mutant F A strain protein further comprises a 5′ untranslated region (UTR), a 3′ UTR, and a poly (A) tail so as to have the structure of 5′UTR-ORF encoding RSV mutant F A strain protein-3′UTR-poly (A) tail, and the ORF encoding RSV mutant F A strain protein has a nucleotide sequence of SEQ ID NO: 6. In one embodiment, the mRNA comprising the ORF encoding RSV mutant F B strain protein further comprises a 5′ untranslated region (UTR), a 3′ UTR, and a poly (A) tail so as to have the structure of 5′UTR-ORF encoding RSV mutant F B strain protein-3′UTR-poly (A) tail, and the ORF encoding RSV mutant F B strain protein has a nucleotide sequence of SEQ ID NO: 2. In another embodiment, the mRNA comprising the ORF encoding hMPV F protein further comprises a 5′ untranslated region (UTR), a 3′ UTR, and a poly (A) tail so as to have the structure of 5′UTR-ORF encoding hMPV F protein-3′UTR-poly (A) tail, and the ORF encoding hMPV F protein has a nucleotide sequence of SEQ ID NO: 10. In one embodiment, the poly (A) tail has a length of 50-250 nucleotides. In some embodiment, the mRNA having the structure of 5′UTR-ORF encoding RSV mutant F A strain protein-3′UTR-poly (A) tail has a nucleotide sequence of SEQ ID NO: 7. In some embodiment, the mRNA having the structure of 5′UTR-ORF encoding RSV mutant F B strain protein-3′UTR-poly (A) tail has a nucleotide sequence of SEQ ID NO: 3. In another embodiment, the mRNA having the structure of 5′UTR-ORF encoding hMPV F protein-3′UTR-poly (A) tail has a nucleotide sequence of SEQ ID NO: 11. In some embodiment, the mRNA having the structure of 5′UTR-ORF encoding RSV mutant F A strain protein-3′UTR-poly (A) tail has a nucleotide sequence having at least 80% identity to SEQ ID NO: 7. In another embodiment, the mRNA having the structure of 5′UTR-ORF encoding RSV mutant F B strain protein-3′UTR-poly (A) tail has a nucleotide sequence having at least 80% identity to SEQ ID NO: 3. In some embodiment, the mRNA having the structure of 5′UTR-ORF encoding hMPV F protein-3′UTR-poly (A) tail has a nucleotide sequence having at least 80% identity to SEQ ID NO: 11.

In some embodiment, the RSV and hMPV vaccine composition according to the present disclosure further comprises a pharmaceutically acceptable carrier. In another embodiment, the pharmaceutically acceptable carrier is a lipid nanoparticle encapsulating the mRNA therein.

The present disclosure also provides a method of inducing immune response against respiratory syncytial virus (RSV) and human metapneumovirus virus (hMPV) comprising administering an effective amount of the RSV and hMPV vaccine composition of the present disclosure to a subject in need thereof.

### BRIEF DESCRIPTION OF THE DRAWINGS

FIG. 1 shows the results of RSV-F IVT.

FIG. 2 shows the results of RSV-F western blot.

FIG. 3 describes the schedule of immunogenicity and challenge test of the combined of RSV mutant F A strain mRNA vaccine and RSV mutant F B strain mRNA vaccine in mice.

FIG. 4 shows the results of RSV pre-fusion specific IgG immunogenic responses. Ver 027 is the combined of RSV mutant F A strain mRNA vaccine and RSV mutant F B strain mRNA vaccine.

FIG. 5 shows neutralizing antibody (nAb) titration to RSV mutant F A strain (a of FIG. 3) and RSV mutant F B strain (b of FIG. 3) strains.

FIG. 6 shows lung viral loads after the combined of RSV mutant F A strain mRNA vaccine and RSV mutant F B strain mRNA vaccine challenge.

FIG. 7 shows direct enumeration of F85-93 epitope-specific CD8+T lymphocytes.

FIG. 8 shows the results of hMPV-F IVT and western blot with FLAG tag.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this disclosure is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this disclosure belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “respiratory syncytial virus (RSV) vaccine composition” refers to a substance used to stimulate the production of antibodies and provide immunity against RSV.

As used herein, the term “respiratory syncytial virus (RSV) and human metapneumovirus virus (hMPV) vaccine composition” refers to a substance used to stimulate the production of antibodies and provide immunity against RSV and hMPV.

As used herein, the term “messenger ribonucleic acid (mRNA)” refers to a single-stranded molecule of RNA that corresponds to the genetic sequence of a gene, and is read by a ribosome in the process of synthesizing a protein.

As used herein, the term “wild type” refers to a gene or gene product which has the characteristics of that gene or gene product when isolated from a naturally occurring source. In contrast, the term “modified” or “mutant” refers to a gene or gene product which displays altered characteristics when compared to the wild type gene or gene product. For example, a mutant DNA polymerase in the present invention is a DNA polymerase which exhibits a reduced uracil detection activity.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

As used herein, the term “open reading frame (ORF)” refers to a nucleotide sequence between the start and stop codons.

As used herein, the term “an open reading frame (ORF) encoding” refers to the nucleotide coding sequence which encodes a polypeptide. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

As used herein, the term “T7 promoter” refers to a promoter derived from a bacteriophage T7.

As used herein, the term “5′ untranslated region (UTR)” refers to a region of an mRNA that is directly upstream (i.e., 5′) from the start codon (the first codon of an mRNA transcript translated by a ribosome) that does not encode a polypeptide.

As used herein, the term “3′ untranslated region (UTR)” refers to a region of an mRNA that is directly downstream (i.e., 3′) from the stop codon (i.e., the codon of an mRNA transcript that signals a termination of translation) that does not encode a polypeptide.

As used herein, the term “poly (A) tail” refers to a long stretch of adenine nucleotides added to the “tail” or 3′ end of the mRNA.

As used herein, the term “pharmaceutically acceptable carrier” refers to any substance or vehicle suitable for delivering a mRNA vaccine to a suitable in vivo or ex vivo site. Such a carrier can include, but is not limited to, an adjuvant, an excipient, a lipid particle, etc.

As used herein, the term “lipid nanoparticle” refers to a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

As used herein, the term “inducing immune response against respiratory syncytial virus (RSV)” refers to providing protective immunity and/or vaccinating a subject against a RSV infection for prophylactic purposes, as well as causing a desired immune response or effect in a subject in need thereof against a RSV infection, for therapeutic purposes. As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all.

An “effective amount” of the RSV vaccine composition (e.g. mRNA) or the RSV and hMPV vaccine composition is provided based, at least in part, on the target tissue, target cell type, means of administration, physical characteristics of the polynucleotide (e.g., size, and extent of modified nucleosides) and other components of the vaccine, and other determinants. In general, an effective amount of the RSV vaccine (e.g., mRNA) or the RSV and hMPV vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

As used herein, the term “X % identity to SEQ ID NO: Y” or “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “nucleotide sequence having at least X % identity to SEQ ID NO: Y and encodes Z protein” means that the nucleotide sequence meets the two different requirements of having at least X % identity to SEQ ID NO: Y and encoding Z protein.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The terms “subject,” “patient,” “individual,” and the like are used interchangeably herein, and refer to any animal, any mammalian subject, or cells thereof whether in vitro or in situ, amenable to the methods described herein. In certain non-limiting embodiments, the patient, subject or individual is a human.

### DETAILED DESCRIPTION

1. The Respiratory Syncytial Virus (RSV) Vaccine Composition, or the RSV and Human Metapneumovirus Virus (hMPV) Vaccine Composition

The present disclosure provides a respiratory syncytial virus (RSV) vaccine composition comprising a messenger ribonucleic acid (mRNA) comprising an open reading frame (ORF) encoding RSV mutant F B strain protein having an amino acid sequence of SEQ ID NO: 1.

In the RSV vaccine composition, the RSV mutant F B strain protein may have an amino acid sequence having at least 80% identity to SEQ ID NO: 1. In another embodiment, the RSV mutant F B strain protein has an amino acid sequence having at least 85% identity to SEQ ID NO: 1. In another embodiment, the RSV mutant F B strain protein has an amino acid sequence having at least 90% identity to SEQ ID NO: 1. In another embodiment, the RSV mutant F B strain protein has an amino acid sequence having at least 95% identity to SEQ ID NO: 1. In another embodiment, the RSV mutant F B strain protein has an amino acid sequence having at least 96% identity to SEQ ID NO: 1. In another embodiment, the RSV mutant F B strain protein has an amino acid sequence having at least 97% identity to SEQ ID NO: 1. In another embodiment, the RSV mutant F B strain protein has an amino acid sequence having at least 98% identity to SEQ ID NO: 1. In another embodiment, the RSV mutant F B strain protein has an amino acid sequence having at least 99% identity to SEQ ID NO: 1.

In the RSV vaccine composition, the mRNA comprising the ORF encoding RSV mutant F B strain protein may further comprise a 5′ untranslated region (UTR), a 3′ UTR, and a poly (A) tail so as to have the structure of 5′UTR-ORF encoding RSV mutant F B strain protein-3′UTR-poly (A) tail, and the ORF encoding RSV mutant F B strain protein may have a nucleotide sequence of SEQ ID NO: 2 (or a nucleotide sequence having at least 80%, 85%, 90%, 95%, 96%, 97%, 98%, 99% or 99% identity to SEQ ID NO: 2).

In the RSV vaccine composition, the mRNA having the structure of 5′UTR-ORF encoding RSV mutant F B strain protein-3′UTR-poly (A) tail may have a nucleotide sequence of SEQ ID NO: 3 (or a nucleotide sequence having at least 80%, 85%, 90%, 95%, 96%, 97%, 98%, 99% or 99% identity to SEQ ID NO: 3).

The present disclosure also provides a RSV vaccine composition comprising a mRNA comprising an ORF encoding RSV mutant F B strain protein having an amino acid sequence of SEQ ID NO: 1, and a mRNA comprising an ORF encoding RSV mutant F A strain protein having an amino acid sequence of SEQ ID NO: 5. The above RSV vaccine composition, comprising both the mRNA encoding the ORF for the RSV mutant F B strain protein and the mRNA encoding the ORF for the RSV mutant F A strain protein, offers greater protection against the RSV mutant F B strain compared to the RSV vaccine composition containing only the mRNA encoding the ORF for the RSV mutant F A strain protein. The latter provides protective efficacy against the RSV mutant F B strain solely based on cross-reactivity.

In the RSV vaccine composition, the RSV mutant F A strain protein may have an amino acid sequence having at least 80% identity to SEQ ID NO: 5. In another embodiment, the RSV mutant F A strain protein has an amino acid sequence having at least 85% identity to SEQ ID NO: 5. In another embodiment, the RSV mutant F A strain protein has an amino acid sequence having at least 90% identity to SEQ ID NO: 5. In another embodiment, the RSV mutant F A strain protein has an amino acid sequence having at least 95% identity to SEQ ID NO: 5. In another embodiment, the RSV mutant F A strain protein has an amino acid sequence having at least 96% identity to SEQ ID NO: 5. In another embodiment, the RSV mutant F A strain protein has an amino acid sequence having at least 97% identity to SEQ ID NO: 5. In another embodiment, the RSV mutant F A strain protein has an amino acid sequence having at least 98% identity to SEQ ID NO: 5. In another embodiment, the RSV mutant F A strain protein has an amino acid sequence having at least 99% identity to SEQ ID NO: 5.

In the RSV vaccine composition, the mRNA comprising the ORF encoding RSV mutant F A strain protein may further comprise a 5′ untranslated region (UTR), a 3′ UTR, and a poly (A) tail so as to have the structure of 5′UTR-ORF encoding RSV mutant F A strain protein-3′UTR-poly (A) tail, and the ORF encoding RSV mutant F A strain protein may have a nucleotide sequence of SEQ ID NO: 6 (or a nucleotide sequence having at least 80%, 85%, 90%, 95%, 96%, 97%, 98%, 99% or 99% identity to SEQ ID NO: 6).

In the RSV vaccine composition, the mRNA having the structure of 5′UTR-ORF encoding RSV mutant F A strain protein-3′UTR-poly (A) tail may have a nucleotide sequence of SEQ ID NO: 7 (or a nucleotide sequence having at least 80%, 85%, 90%, 95%, 96%, 97%, 98%, 99% or 99% identity to SEQ ID NO: 7).

The present disclosure also provides a respiratory syncytial virus (RSV) and human metapneumovirus virus (hMPV) vaccine composition comprising a messenger ribonucleic acid (mRNA) comprising an open reading frame (ORF) encoding RSV mutant F A strain protein having an amino acid sequence of SEQ ID NO: 5, a mRNA comprising an ORF encoding RSV mutant F B strain protein having an amino acid sequence of SEQ ID NO: 1, and a mRNA comprising an ORF encoding hMPV F protein having an amino acid sequence of SEQ ID NO: 9.

In the RSV and hMPV vaccine composition, the RSV mutant F A strain protein may have an amino acid sequence having at least 80% identity to SEQ ID NO: 5. In another embodiment, the RSV mutant F A strain protein has an amino acid sequence having at least 85% identity to SEQ ID NO: 5. In another embodiment, the RSV mutant F A strain protein has an amino acid sequence having at least 90% identity to SEQ ID NO: 5. In another embodiment, the RSV mutant F A strain protein has an amino acid sequence having at least 95% identity to SEQ ID NO: 5. In another embodiment, the RSV mutant F A strain protein has an amino acid sequence having at least 96% identity to SEQ ID NO: 5. In another embodiment, the RSV mutant F A strain protein has an amino acid sequence having at least 97% identity to SEQ ID NO: 5. In another embodiment, the RSV mutant F A strain protein has an amino acid sequence having at least 98% identity to SEQ ID NO: 5. In another embodiment, the RSV mutant F A strain protein has an amino acid sequence having at least 99% identity to SEQ ID NO: 5.

In the RSV and hMPV vaccine composition, the RSV mutant F B strain protein may have an amino acid sequence having at least 80% identity to SEQ ID NO: 1. In another embodiment, the RSV mutant F B strain protein has an amino acid sequence having at least 85% identity to SEQ ID NO: 1. In another embodiment, the RSV mutant F B strain protein has an amino acid sequence having at least 90% identity to SEQ ID NO: 1. In another embodiment, the RSV mutant F B strain protein has an amino acid sequence having at least 95% identity to SEQ ID NO: 1. In another embodiment, the RSV mutant F B strain protein has an amino acid sequence having at least 96% identity to SEQ ID NO: 1. In another embodiment, the RSV mutant F B strain protein has an amino acid sequence having at least 97% identity to SEQ ID NO: 1. In another embodiment, the RSV mutant F B strain protein has an amino acid sequence having at least 98% identity to SEQ ID NO: 1. In another embodiment, the RSV mutant F B strain protein has an amino acid sequence having at least 99% identity to SEQ ID NO: 1.

In the RSV and hMPV vaccine composition, the hMPV F protein may have an amino acid sequence having at least 80% identity to SEQ ID NO: 9. In another embodiment, the hMPV F protein has an amino acid sequence having at least 85% identity to SEQ ID NO: 9. In another embodiment, the hMPV F protein has an amino acid sequence having at least 90% identity to SEQ ID NO: 9. In another embodiment, the hMPV F protein has an amino acid sequence having at least 95% identity to SEQ ID NO: 9. In another embodiment, the hMPV F protein has an amino acid sequence having at least 96% identity to SEQ ID NO: 9. In another embodiment, the hMPV F protein has an amino acid sequence having at least 97% identity to SEQ ID NO: 9. In another embodiment, the hMPV F protein has an amino acid sequence having at least 98% identity to SEQ ID NO: 9. In another embodiment, the hMPV F protein has an amino acid sequence having at least 99% identity to SEQ ID NO: 9.

In one embodiment, the ORF encoding RSV mutant F A strain protein has a nucleotide sequence of SEQ ID NO: 6, the ORF encoding RSV mutant F B strain protein has a nucleotide sequence of SEQ ID NO: 2, and the ORF encoding hMPV F protein has a nucleotide sequence of SEQ ID NO: 10. In another embodiment, the mRNA comprising the ORF encoding RSV mutant F A strain protein further comprises a 5′ untranslated region (UTR), a 3′ UTR, and a poly (A) tail so as to have the structure of 5′UTR-ORF encoding RSV mutant F A strain protein-3′UTR-poly (A) tail, and the ORF encoding RSV mutant F A strain protein has a nucleotide sequence of SEQ ID NO: 6. In one embodiment, the mRNA comprising the ORF encoding RSV mutant F B strain protein further comprises a 5′ untranslated region (UTR), a 3′ UTR, and a poly (A) tail so as to have the structure of 5′UTR-ORF encoding RSV mutant F B strain protein-3′UTR-poly (A) tail, and the ORF encoding RSV mutant F B strain protein has a nucleotide sequence of SEQ ID NO: 2. In another embodiment, the mRNA comprising the ORF encoding hMPV F protein further comprises a 5′ untranslated region (UTR), a 3′ UTR, and a poly (A) tail so as to have the structure of 5′UTR-ORF encoding hMPV F protein-3′UTR-poly (A) tail, and the ORF encoding hMPV F protein has a nucleotide sequence of SEQ ID NO: 10.

In the RSV and hMPV vaccine composition, the ORF encoding RSV mutant F A strain protein may have a nucleotide sequence having at least 80% identity to SEQ ID NO: 6. In another embodiment, the ORF encoding RSV mutant F A strain protein may have a nucleotide sequence having at least 85% identity to SEQ ID NO: 6. In another embodiment, the ORF encoding RSV mutant F A strain protein may have a nucleotide sequence having at least 90% identity to SEQ ID NO: 6. In another embodiment, the ORF encoding RSV mutant F A strain protein may have a nucleotide sequence having at least 95% identity to SEQ ID NO: 6. In another embodiment, the ORF encoding RSV mutant F A strain protein may have a nucleotide sequence having at least 96% identity to SEQ ID NO: 6. In another embodiment, the ORF encoding RSV mutant F A strain protein may have a nucleotide sequence having at least 97% identity to SEQ ID NO: 6. In another embodiment, the ORF encoding RSV mutant F A strain protein may have a nucleotide sequence having at least 98% identity to SEQ ID NO: 6. In another embodiment, the ORF encoding RSV mutant F A strain protein may have a nucleotide sequence having at least 99% identity to SEQ ID NO: 6.

In the RSV and hMPV vaccine composition, the ORF encoding RSV mutant F B strain protein may have a nucleotide sequence having at least 80% identity to SEQ ID NO: 2. In another embodiment, the ORF encoding RSV mutant F B strain protein may have a nucleotide sequence having at least 85% identity to SEQ ID NO: 2. In another embodiment, the ORF encoding RSV mutant F B strain protein may have a nucleotide sequence having at least 90% identity to SEQ ID NO: 2. In another embodiment, the ORF encoding RSV mutant F B strain protein may have a nucleotide sequence having at least 95% identity to SEQ ID NO: 2. In another embodiment, the ORF encoding RSV mutant F B strain protein may have a nucleotide sequence having at least 96% identity to SEQ ID NO: 2. In another embodiment, the ORF encoding RSV mutant F B strain protein may have a nucleotide sequence having at least 97% identity to SEQ ID NO: 2. In another embodiment, the ORF encoding RSV mutant F B strain protein may have a nucleotide sequence having at least 98% identity to SEQ ID NO: 2. In another embodiment, the ORF encoding RSV mutant F B strain protein may have a nucleotide sequence having at least 99% identity to SEQ ID NO: 2.

In the RSV and hMPV vaccine composition, the ORF encoding hMPV F protein may have a nucleotide sequence having at least 80% identity to SEQ ID NO: 10. In another embodiment, the ORF encoding hMPV F protein may have a nucleotide sequence having at least 85% identity to SEQ ID NO: 10. In another embodiment, the ORF encoding hMPV F protein may have a nucleotide sequence having at least 90% identity to SEQ ID NO: 10. In another embodiment, the ORF encoding hMPV F protein may have a nucleotide sequence having at least 95% identity to SEQ ID NO: 10. In another embodiment, the ORF encoding hMPV F protein may have a nucleotide sequence having at least 96% identity to SEQ ID NO: 10. In another embodiment, the ORF encoding hMPV F protein may have a nucleotide sequence having at least 97% identity to SEQ ID NO: 10. In another embodiment, the ORF encoding hMPV F protein may have a nucleotide sequence having at least 98% identity to SEQ ID NO: 10. In another embodiment, the ORF encoding hMPV F protein may have a nucleotide sequence having at least 99% identity to SEQ ID NO: 10.

In one embodiment, the poly (A) tail has a length of 50-250 nucleotides. In some embodiment, the mRNA having the structure of 5′UTR-ORF encoding RSV mutant F A strain protein-3′UTR-poly (A) tail has a nucleotide sequence of SEQ ID NO: 7. In some embodiment, the mRNA having the structure of 5′UTR-ORF encoding RSV mutant F B strain protein-3′UTR-poly (A) tail has a nucleotide sequence of SEQ ID NO: 3. In another embodiment, the mRNA having the structure of 5′UTR-ORF encoding hMPV F protein-3′UTR-poly (A) tail has a nucleotide sequence of SEQ ID NO: 11. In some embodiment, the mRNA having the structure of 5′UTR-ORF encoding RSV mutant F A strain protein-3′UTR-poly (A) tail has a nucleotide sequence having at least 80% identity to SEQ ID NO: 7 (or a nucleotide sequence having at least 85%, 90%, 95%, 96%, 97%, 98%, 99% or 99% identity to SEQ ID NO: 7). In another embodiment, the mRNA having the structure of 5′UTR-ORF encoding RSV mutant F B strain protein-3′UTR-poly (A) tail has a nucleotide sequence having at least 80% identity to SEQ ID NO: 3 (or a nucleotide sequence having at least 85%, 90%, 95%, 96%, 97%, 98%, 99% or 99% identity to SEQ ID NO: 3). In some embodiment, the mRNA having the structure of 5′UTR-ORF encoding hMPV F protein-3′UTR-poly (A) tail has a nucleotide sequence having at least 80% identity to SEQ ID NO: 11 (or a nucleotide sequence having at least 85%, 90%, 95%, 96%, 97%, 98%, 99% or 99% identity to SEQ ID NO: 11).

In one embodiment, the poly (A) tail has a length of 50-250 nucleotides. In another embodiment, the poly (A) tail has a length of 100-200 nucleotides. In another embodiment, the poly (A) tail has a length of 110-150 nucleotides. In another embodiment, the poly (A) tail has a length of 115-125 nucleotides. In another embodiment, the poly (A) tail has a length of 116-124 nucleotides. In another embodiment, the poly (A) tail has a length of 117-123 nucleotides. In another embodiment, the poly (A) tail has a length of 118-122 nucleotides. In another embodiment, the poly (A) tail has a length of 119-122 nucleotides. In another embodiment, the poly (A) tail has a length of 115 nucleotides. In another embodiment, the poly (A) tail has a length of 116 nucleotides. In another embodiment, the poly (A) tail has a length of 117 nucleotides. In another embodiment, the poly (A) tail has a length of 118 nucleotides. In another embodiment, the poly (A) tail has a length of 119 nucleotides. In another embodiment, the poly (A) tail has a length of 120 nucleotides. In another embodiment, the poly (A) tail has a length of 121 nucleotides. In another embodiment, the poly (A) tail has a length of 122 nucleotides. In another embodiment, the poly (A) tail has a length of 123 nucleotides. In another embodiment, the poly (A) tail has a length of 124 nucleotides. In another embodiment, the poly (A) tail has a length of 125 nucleotides.

In one embodiment, the mRNA of the present disclosure may comprise at least one chemical modification selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In another embodiment, the chemical modification is in the 5-position of the uracil. In another embodiment, the chemical modification is a N1-methylpseudouridine. In another embodiments, the chemical modification is a N1-ethylpseudouridine.

In one embodiment, the RSV vaccine composition further comprises a pharmaceutically acceptable carrier. In another embodiment, the pharmaceutically acceptable carrier may include any substance or vehicle suitable for delivering a mRNA vaccine to a suitable in vivo or ex vivo site. Such a carrier can include, but is not limited to, an adjuvant, an excipient, a lipid particle, etc. The lipid nanoparticle may be a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In one embodiment, the lipid nanoparticle comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), (ii) a neutral lipid selected from DSPC, DPPC, POPC, DOPE and SM, (iii) a sterol, e.g., cholesterol, and (iv) a PEG-lipid, e.g., PEG-DMG or PEG-CDMA, in a molar ratio of about 20-60% cationic lipid:5-25% neutral lipid:25-55% sterol; 0.5-15% PEG-lipid.

In one embodiment, the lipid nanoparticle includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about to about 65%, from about 45 to about 65%, about 60%, about 57.5%, about 50% or about 40% on a molar basis.

In one embodiment, the lipid nanoparticle includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), and PEG-cDMA.

In one embodiment, the lipid nanoparticle includes 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the lipid nanoparticle include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the lipid nanoparticle includes 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the lipid nanoparticle includes about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the lipid nanoparticle includes about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the RSV vaccine composition or the RSV and hMPV vaccine composition of the present disclosure may be delivered, localized and/or concentrated in a specific location using the delivery methods described as follows. As a non-limiting example, a subject may be administered an empty polymeric particle prior to, simultaneously with or after delivering the RSV vaccine composition of the present disclosure to the subject. The empty polymeric particle undergoes a change in volume once in contact with the subject and becomes lodged, embedded, immobilized or entrapped at a specific location in the subject.

In another embodiment, the RSV vaccine composition of the present disclosure may be formulated in an active substance release system. For instance, the active substance release system may comprise at least one nanoparticle bonded to an oligonucleotide inhibitor strand which is hybridized with a catalytically active nucleic acid and a compound bonded to at least one substrate molecule bonded to a therapeutically active substance (e.g., polynucleotides described herein), where the therapeutically active substance is released by the cleavage of the substrate molecule by the catalytically active nucleic acid.

In another embodiment, the RSV vaccine composition or the RSV and hMPV vaccine composition of the present disclosure may be formulated in a nanoparticle comprising an inner core comprising a non-cellular material and an outer surface comprising a cellular membrane. The cellular membrane may be derived from a cell or a membrane derived from a virus.

In another embodiment, the RSV vaccine composition or the RSV and hMPV vaccine composition of the present disclosure may be formulated in porous nanoparticle-supported lipid bilayers (protocells).

In another embodiment, the RSV vaccine composition or the RSV and hMPV vaccine composition of the present disclosure may be formulated in polymeric nanoparticles which have a high glass transition temperature.

In another embodiment, the RSV vaccine composition or the RSV and hMPV vaccine composition of the present disclosure may be formulated in nanoparticles used in imaging. As a non-limiting example, the liposome may comprise gadolinium(III)2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component.

The nanoparticles of the present disclosure may further include nutrients such as, but not limited to, those which deficiencies can lead to health hazards from anemia to neural tube defects. As a non-limiting example, the nutrient may be iron in the form of ferrous, ferric salts or elemental iron, iodine, folic acid, vitamins or micronutrients.

In another embodiment, the RSV vaccine composition or the RSV and hMPV vaccine composition of the present disclosure may be formulated in a swellable nanoparticle.

In another embodiment, the RSV vaccine composition or the RSV and hMPV vaccine composition of the present disclosure may be formulated in polyanhydride nanoparticles.

The nanoparticles and microparticles of the present disclosure may be geometrically engineered to modulate macrophage and/or the immune response. In some embodiments, the geometrically engineered particles may have varied shapes, sizes and/or surface charges in order to incorporated the polynucleotides of the present disclosure for targeted delivery such as, but not limited to, pulmonary delivery. Other physical features the geometrically engineering particles may have include, but are not limited to, fenestrations, angled arms, asymmetry and surface roughness, charge which can alter the interactions with cells and tissues.

In another embodiment, the nanoparticles of the present disclosure may be water soluble nanoparticles. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments, the nanoparticles of the present disclosure are stealth nanoparticles or target-specific stealth nanoparticles. In some embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle of the present disclosure may be a nanoparticle-nucleic acid hybrid structure having a high density nucleic acid layer. The nanoparticle of the present disclosure may comprise a nucleic acid such as, but not limited to, polynucleotides described herein and/or known in the art.

In one embodiment, at least one of the nanoparticles of the present disclosure may be embedded in in the core a nanostructure or coated with a low density porous 3-D structure or coating which is capable of carrying or associating with at least one payload within or on the surface of the nanostructure.

In one embodiment, the pharmaceutically acceptable carrier is a lipid nanoparticle encapsulating the mRNAs of the present disclosure therein.

2. The Method of Inducing Immune Response Against Respiratory Syncytial Virus (RSV), or the Method of Inducing Immune Response Against RSV and Human Metapneumovirus Virus (hMPV)

The present disclosure also provides a method of inducing immune response against respiratory syncytial virus (RSV) comprising administering an effective amount of the RSV vaccine composition of the present disclosure a subject in need thereof.

The present disclosure also provides a method of inducing immune response against RSV and human metapneumovirus virus (hMPV) comprising administering an effective amount of the RSV and hMPV vaccine composition of the present disclosure a subject in need thereof.

In one embodiment, the effective amount of the RSV vaccine composition or the RSV and hMPV vaccine composition (e.g. mRNA) is provided based, at least in part, on the target tissue, target cell type, means of administration, physical characteristics of the polynucleotide (e.g., size, and extent of modified nucleosides) and other components of the vaccine, and other determinants. In general, an effective amount of the RSV vaccine the RSV and hMPV vaccine composition (e.g., mRNA) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

Administration of an effective amount (immunogenically effective amount) of the RSV vaccine composition the RSV and hMPV vaccine composition is typically intramuscular or subcutaneous. Thus, the RSV vaccine composition is typically formulated for intramuscular or subcutaneous injection, and for the purposes of the invention formulated without adjuvants, preferably without any adjuvant. However other modes of administration, such as intravenous, cutaneous, intradermal or nasal can be envisaged as well. For intravenous, cutaneous or subcutaneous injection, the adenovirus vector will be in the form of a parenterally acceptable aqueous solution which is pyrogen-free and has suitable pH, isotonicity and stability. Likewise, the isolated envelope polypeptide will be in the form of a parenterally acceptable solution having a suitable pH, isotonicity, and stability. Those of ordinary skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In a particular embodiment, an effective amount (immunogenically effective amount) of the RSV vaccine composition the RSV and hMPV vaccine composition is administered via intramuscular administration. Intramuscular administration can be achieved by using a needle to inject a suspension of the adenovirus vectors and/or envelope polypeptides. An alternative is the use of a needleless injection device to administer the composition (using, e.g., Biojector™) or a freeze-dried powder containing the vaccine.

In one embodiment, the priming immunization and/or the boosting administration, preferably both the priming and boosting administration, further comprise administering one or more adenovirus vectors that encode one or more further RSV antigens or RSV and hMPV antigens.

The timing for administering priming and boosting immunizations is not particularly limited. For example, a vaccine composition can be administered for priming immunization, and re-administered prior to administration of a vaccine composition for boosting immunization. Further administrations of a vaccine composition for further boosting immunizations are also contemplated. In certain embodiments, a booster vaccine is first administered about 1-12 weeks, e.g., 1, 2, 3, 4, 5, 6, 7, 8, 9, 10, 11, or 12 weeks after a primer vaccine is initially administered. In other embodiments, a booster vaccine is first administered about 12-52 weeks, e.g., about 12, 14, 16, 18, 20, 22, 24, 26, 28, 30, 32, 34, 36, 38, 40, 42, 44, 46, 48, 50, or 52 weeks after a primer vaccine is initially administered. One of ordinary skill in the art will be able to vary the exact timing of the priming and boosting vaccines, frequency of administration thereof, dosage thereof, etc., based upon the teachings herein and general knowledge in the art.

In one embodiment, the RSV vaccine composition or the RSV and hMPV vaccine composition may comprise the first and second mRNAs described herein, formulated in a lipid nanoparticle comprising MC3, Cholesterol, DSPC and PEG2000-DMG, the buffer trisodium citrate, sucrose and water for injection. As a non-limiting example, the composition may comprise 2.0 mg/mL of drug substance, 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In one embodiment, the method of inducing immune response against RSV comprises administering an effective amount of the RSV vaccine composition of the present disclosure to a subject in need thereof. In the RSV vaccine composition, the mRNA having the structure of 5′UTR-ORF encoding RSV mutant F B strain protein-3′UTR-poly (A) tail may have a nucleotide sequence of SEQ ID NO: 3 (or a nucleotide sequence having at least 80%, 85%, 90%, 95%, 96%, 97%, 98%, 99% or 99% identity to SEQ ID NO: 3).

In another embodiment, the RSV vaccine composition comprises a mRNA having the structure of 5′UTR-ORF encoding RSV mutant F B strain protein-3′UTR-poly (A) tail may have a nucleotide sequence of SEQ ID NO: 3 (or a nucleotide sequence having at least 80%, 85%, 90%, 95%, 96%, 97%, 98%, 99% or 99% identity to SEQ ID NO: 3), and a mRNA having the structure of 5′UTR-ORF encoding RSV mutant F A strain protein-3′UTR-poly (A) tail may have a nucleotide sequence of SEQ ID NO: 7 (or a nucleotide sequence having at least 80%, 85%, 90%, 95%, 96%, 97%, 98%, 99% or 99% identity to SEQ ID NO: 7).

In another embodiment, the method of inducing immune response against RSV and hMPV comprises administering an effective amount of the RSV and hMPV vaccine composition of the present disclosure to a subject in need thereof. In the RSV and hMPV vaccine composition, the mRNA having the structure of 5′UTR-ORF encoding RSV mutant F A strain protein-3′UTR-poly (A) tail may have a nucleotide sequence of SEQ ID NO: 7 (or a nucleotide sequence having at least 80%, 85%, 90%, 95%, 96%, 97%, 98%, 99% or 99% identity to SEQ ID NO: 7). In addition, the mRNA having the structure of 5′UTR-ORF encoding RSV mutant F B strain protein-3′UTR-poly (A) tail may have a nucleotide sequence of SEQ ID NO: 3 (or a nucleotide sequence having at least 80%, 85%, 90%, 95%, 96%, 97%, 98%, 99% or 99% identity to SEQ ID NO: 3). Moreover, the mRNA having the structure of 5′UTR-ORF encoding hMPV protein-3′UTR-poly (A) tail may have a nucleotide sequence of SEQ ID NO: 11 (or a nucleotide sequence having at least 80%, 85%, 90%, 95%, 96%, 97%, 98%, 99% or 99% identity to SEQ ID NO: 11).

### 3. Sequence Information

1) Protein sequence of the ORF encoded in RSV mutant F B strain mRNA
SEQ ID NO: 1
MELLIHRSSAIFLTLAINALYLTSSQNITEEFYQSTCSAVSRGYLSALRTGWYTSVITIELS
NIKETKCNGTDTKVKLIKQELDKYKNAVTELQLLMQNTPAVNNRARREAPQYMNYTIN
TTKNLNVSISKKRKRRFLGFLLGVGSAIASGIAVCKVLHLEGEVNKIKNALQLTNKAVVS
LSNGVSVLTFRVLDLKNYINNQLLPMLNRQSCRISNIETVIEFQQKNSRLLEITREFSVNA
GVTTPLSTYMLTNSELLSLINDMPITNDQKKLMSSNVQIVRQQSYSIMCIIKEEVLAYVV
QLPIYGVIDTPCWKLHTSPLCTTNIKEGSNICLTRTDRGWYCDNAGSVSFFPQADTCKVQ
SNRVFCDTMNSLTLPSEVSLCNTDIFNSKYDCKIMTSKTDISSSVITSLGAIVSCYGKTKC
TASNKNRGIIKTFSNGCDYVSNKGVDTVSVGNTLYYVNKLEGKNLYVKGEPIINYYDPL
VFPSDEFDASISQVNEKINQSLAFIRRSDELLHNVNTGKSTTNIMITAIIIVIIVVLLSLIAIGL
LLYCKAKNTPVTLSKDQLSGINNIAFSK
2) RSV mutant F B strain mRNA sequence (ORF)
SEQ ID NO: 2
AUGGAACUGCUGAUCCACAGAUCCAGCGCUAUUUUCCUGACACUGGCCAUCAAUG
CCCUGUACCUGACAAGCAGCCAGAACAUCACAGAGGAAUUCUACCAGAGUACCUG
UAGCGCUGUGUCUCGGGGAUACCUGAGCGCCCUGCGGACCGGUUGGUACACCAGC
GUGAUCACAAUCGAGCUGAGUAACAUCAAGGAGACAAAAUGCAAUGGCACUGAC
ACCAAGGUGAAACUUAUCAAACAGGAGCUGGAUAAGUACAAGAAUGCCGUGACA
GAACUGCAGCUGCUGAUGCAGAAUACCCCCGCCGUGAACAACAGAGCCAGACGGG
AAGCCCCUCAAUACAUGAACUACACAAUCAACACCACAAAGAACCUGAACGUGUC
UAUCUCCAAGAAGCGCAAGCGGAGAUUCCUGGGCUUCCUGUUGGGAGUGGGCUC
UGCCAUCGCCAGCGGCAUCGCAGUGUGCAAGGUGCUGCACCUGGAAGGAGAAGUG
AACAAGAUCAAGAAUGCCCUGCAGCUGACCAACAAGGCCGUGGUCUCUCUGAGCA
AUGGCGUGAGCGUGCUGACCUUUAGAGUGCUGGACCUGAAGAACUACAUCAACA
ACCAGCUGCUGCCUAUGCUGAACAGACAGUCUUGUAGAAUCAGCAACAUCGAAAC
CGUGAUCGAGUUCCAGCAGAAGAACUCCAGGCUGCUGGAAAUCACAAGAGAGUU
UAGCGUGAACGCCGGCGUCACCACCCCCCUGAGCACCUACAUGCUGACCAACAGC
GAGCUGCUGAGCCUGAUUAACGACAUGCCUAUCACCAAUGACCAGAAAAAGCUAA
UGAGCAGCAAUGUGCAAAUCGUGCGGCAGCAGUCCUACAGCAUCAUGUGCAUCAU
CAAGGAAGAGGUCCUGGCCUACGUGGUACAACUGCCUAUCUACGGCGUGAUCGAC
ACCCCUUGUUGGAAGCUCCAUACCAGCCCUCUGUGCACAACAAACAUCAAAGAAG
GCAGCAAUAUCUGCCUGACAAGAACAGAUAGAGGCUGGUAUUGUGACAACGCUG
GCUCCGUCUCCUUCUUCCCUCAGGCCGACACCUGCAAGGUGCAGAGCAACCGCGU
GUUCUGCGAUACCAUGAACAGCUUAACACUGCCCAGCGAGGUGUCUCUCUGUAAC
ACCGACAUCUUCAACUCUAAAUACGACUGCAAGAUCAUGACCUCUAAGACUGAUA
UCAGCAGCAGCGUGAUUACCUCCCUGGGCGCCAUCGUGUCUUGCUACGGCAAGAC
AAAGUGCACCGCCAGCAACAAGAACCGGGGCAUCAUCAAGACCUUCAGCAACGGC
UGCGACUACGUGAGCAACAAGGGCGUGGACACCGUUAGCGUGGGCAACACACUGU
AUUACGUAAAUAAACUUGAGGGCAAGAAUCUGUACGUGAAGGGCGAGCCUAUCA
UCAACUACUACGACCCACUGGUGUUUCCAUCUGAUGAGUUCGACGCCUCCAUCUC
CCAGGUGAACGAGAAGAUCAACCAGAGCCUGGCUUUUAUCAGAAGAAGCGAUGA
GCUGCUGCACAACGUGAAUACCGGAAAAAGCACCACCAACAUCAUGAUCACCGCC
AUCAUUAUCGUUAUCAUCGUGGUGCUGCUGAGUCUGAUCGCCAUUGGACUGCUG
CUGUAUUGCAAAGCCAAGAACACCCCCGUGACCCUGAGCAAGGACCAGCUGAGCG
GCAUCAACAACAUAGCUUUCUCCAAG
3) RSV mutant F B strain mRNA sequence (5′-UTR-ORF-3′UTR-Poly(A) tail)
SEQ ID NO: 3
AGGCCGGCACUCUUCUGGUCCCCACAGACUCAGAGAGAACCCGCCGCCACCAUGG
AACUGCUGAUCCACAGAUCCAGCGCUAUUUUCCUGACACUGGCCAUCAAUGCCCU
GUACCUGACAAGCAGCCAGAACAUCACAGAGGAAUUCUACCAGAGUACCUGUAGC
GCUGUGUCUCGGGGAUACCUGAGCGCCCUGCGGACCGGUUGGUACACCAGCGUGA
UCACAAUCGAGCUGAGUAACAUCAAGGAGACAAAAUGCAAUGGCACUGACACCA
AGGUGAAACUUAUCAAACAGGAGCUGGAUAAGUACAAGAAUGCCGUGACAGAAC
UGCAGCUGCUGAUGCAGAAUACCCCCGCCGUGAACAACAGAGCCAGACGGGAAGC
CCCUCAAUACAUGAACUACACAAUCAACACCACAAAGAACCUGAACGUGUCUAUC
UCCAAGAAGCGCAAGCGGAGAUUCCUGGGCUUCCUGUUGGGAGUGGGCUCUGCCA
UCGCCAGCGGCAUCGCAGUGUGCAAGGUGCUGCACCUGGAAGGAGAAGUGAACA
AGAUCAAGAAUGCCCUGCAGCUGACCAACAAGGCCGUGGUCUCUCUGAGCAAUGG
CGUGAGCGUGCUGACCUUUAGAGUGCUGGACCUGAAGAACUACAUCAACAACCAG
CUGCUGCCUAUGCUGAACAGACAGUCUUGUAGAAUCAGCAACAUCGAAACCGUGA
UCGAGUUCCAGCAGAAGAACUCCAGGCUGCUGGAAAUCACAAGAGAGUUUAGCG
UGAACGCCGGCGUCACCACCCCCCUGAGCACCUACAUGCUGACCAACAGCGAGCU
GCUGAGCCUGAUUAACGACAUGCCUAUCACCAAUGACCAGAAAAAGCUAAUGAGC
AGCAAUGUGCAAAUCGUGCGGCAGCAGUCCUACAGCAUCAUGUGCAUCAUCAAGG
AAGAGGUCCUGGCCUACGUGGUACAACUGCCUAUCUACGGCGUGAUCGACACCCC
UUGUUGGAAGCUCCAUACCAGCCCUCUGUGCACAACAAACAUCAAAGAAGGCAGC
AAUAUCUGCCUGACAAGAACAGAUAGAGGCUGGUAUUGUGACAACGCUGGCUCC
GUCUCCUUCUUCCCUCAGGCCGACACCUGCAAGGUGCAGAGCAACCGCGUGUUCU
GCGAUACCAUGAACAGCUUAACACUGCCCAGCGAGGUGUCUCUCUGUAACACCGA
CAUCUUCAACUCUAAAUACGACUGCAAGAUCAUGACCUCUAAGACUGAUAUCAGC
AGCAGCGUGAUUACCUCCCUGGGCGCCAUCGUGUCUUGCUACGGCAAGACAAAGU
GCACCGCCAGCAACAAGAACCGGGGCAUCAUCAAGACCUUCAGCAACGGCUGCGA
CUACGUGAGCAACAAGGGCGUGGACACCGUUAGCGUGGGCAACACACUGUAUUAC
GUAAAUAAACUUGAGGGCAAGAAUCUGUACGUGAAGGGCGAGCCUAUCAUCAAC
UACUACGACCCACUGGUGUUUCCAUCUGAUGAGUUCGACGCCUCCAUCUCCCAGG
UGAACGAGAAGAUCAACCAGAGCCUGGCUUUUAUCAGAAGAAGCGAUGAGCUGC
UGCACAACGUGAAUACCGGAAAAAGCACCACCAACAUCAUGAUCACCGCCAUCAU
UAUCGUUAUCAUCGUGGUGCUGCUGAGUCUGAUCGCCAUUGGACUGCUGCUGUA
UUGCAAAGCCAAGAACACCCCCGUGACCCUGAGCAAGGACCAGCUGAGCGGCAUC
AACAACAUAGCUUUCUCCAAGUGACUCGAGUAAGCUGGAGCCUCGGUGGCCAUGC
UUCUUGCCCCUUGGGCCUCCCCCCAGCCCCUCCUCCCCUUCCUGCACCCGUACCCC
CGUGGUCUUUGAAUAAAGUCUGAGUGGGCGGCAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAA
4) Sequence of pUC57 plasmid encoding RSV mutant F B strain mRNA sequence
(mRNA sequence has been underlined)
SEQ ID NO: 4
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GTGAATTCGAGCTCGGTACCTCGCGAATGCATCTAGATTCTAGATTAATACGACTCA
CTATAAGGCCGGCACTCTTCTGGTCCCCACAGACTCAGAGAGAACCCGCCGCCACC
ATGGAACTGCTGATCCACAGATCCAGCGCTATTTTCCTGACACTGGCCATCAATGCC
CTGTACCTGACAAGCAGCCAGAACATCACAGAGGAATTCTACCAGAGTACCTGTAG
CGCTGTGTCTCGGGGATACCTGAGCGCCCTGCGGACCGGTTGGTACACCAGCGTGAT
CACAATCGAGCTGAGTAACATCAAGGAGACAAAATGCAATGGCACTGACACCAAGG
TGAAACTTATCAAACAGGAGCTGGATAAGTACAAGAATGCCGTGACAGAACTGCAG
CTGCTGATGCAGAATACCCCCGCCGTGAACAACAGAGCCAGACGGGAAGCCCCTCA
ATACATGAACTACACAATCAACACCACAAAGAACCTGAACGTGTCTATCTCCAAGA
AGCGCAAGCGGAGATTCCTGGGCTTCCTGTTGGGAGTGGGCTCTGCCATCGCCAGCG
GCATCGCAGTGTGCAAGGTGCTGCACCTGGAAGGAGAAGTGAACAAGATCAAGAAT
GCCCTGCAGCTGACCAACAAGGCCGTGGTCTCTCTGAGCAATGGCGTGAGCGTGCT
GACCTTTAGAGTGCTGGACCTGAAGAACTACATCAACAACCAGCTGCTGCCTATGCT
GAACAGACAGTCTTGTAGAATCAGCAACATCGAAACCGTGATCGAGTTCCAGCAGA
AGAACTCCAGGCTGCTGGAAATCACAAGAGAGTTTAGCGTGAACGCCGGCGTCACC
ACCCCCCTGAGCACCTACATGCTGACCAACAGCGAGCTGCTGAGCCTGATTAACGA
CATGCCTATCACCAATGACCAGAAAAAGCTAATGAGCAGCAATGTGCAAATCGTGC
GGCAGCAGTCCTACAGCATCATGTGCATCATCAAGGAAGAGGTCCTGGCCTACGTG
GTACAACTGCCTATCTACGGCGTGATCGACACCCCTTGTTGGAAGCTCCATACCAGC
CCTCTGTGCACAACAAACATCAAAGAAGGCAGCAATATCTGCCTGACAAGAACAGA
TAGAGGCTGGTATTGTGACAACGCTGGCTCCGTCTCCTTCTTCCCTCAGGCCGACAC
CTGCAAGGTGCAGAGCAACCGCGTGTTCTGCGATACCATGAACAGCTTAACACTGC
CCAGCGAGGTGTCTCTCTGTAACACCGACATCTTCAACTCTAAATACGACTGCAAGA
TCATGACCTCTAAGACTGATATCAGCAGCAGCGTGATTACCTCCCTGGGCGCCATCG
TGTCTTGCTACGGCAAGACAAAGTGCACCGCCAGCAACAAGAACCGGGGCATCATC
AAGACCTTCAGCAACGGCTGCGACTACGTGAGCAACAAGGGCGTGGACACCGTTAG
CGTGGGCAACACACTGTATTACGTAAATAAACTTGAGGGCAAGAATCTGTACGTGA
AGGGCGAGCCTATCATCAACTACTACGACCCACTGGTGTTTCCATCTGATGAGTTCG
ACGCCTCCATCTCCCAGGTGAACGAGAAGATCAACCAGAGCCTGGCTTTTATCAGA
AGAAGCGATGAGCTGCTGCACAACGTGAATACCGGAAAAAGCACCACCAACATCAT
GATCACCGCCATCATTATCGTTATCATCGTGGTGCTGCTGAGTCTGATCGCCATTGG
ACTGCTGCTGTATTGCAAAGCCAAGAACACCCCCGTGACCCTGAGCAAGGACCAGC
TGAGCGGCATCAACAACATAGCTTTCTCCAAGTGACTCGAGTAAGCTGGAGCCTCGG
TGGCCATGCTTCTTGCCCCTTGGGCCTCCCCCCAGCCCCTCCTCCCCTTCCTGCACCC
GTACCCCCGTGGTCTTTGAATAAAGTCTGAGTGGGCGGCAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAATGAAG
AGCATCGGATCCCGGGCCCGTCGACTGCAGAGGCCTGCATGCAAGCTTGGCGTAAT
CATGGTCATAGCTGTTTCCTGTGTGAAATTGTTATCCGCTCACAATTCCACACAACAT
ACGAGCCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGGCGCTCTT
CCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGA
AAGAACATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGCT
CAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGAAGAAC
AGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACC
TATCTCAGCGATCTGTCTATTTCGTTCATCCATAGTTGCCTGACTCCCCGTCGTGTAG
ATAACTACGATACGGGAGGGCTTACCATCTGGCCCCAGTGCTGCAATGATACCGCG
AGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCA
TTGCTACAGGCATCGTGGTGTCACGCTCGTCGTTTGGTATGGCTTCATTCAGCTCCGG
TTCCCAACGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTATCACTCATG
GTTATGGCAGCACTGCATAATTCTCTTACTGTCATGCCATCCGTAAGATGCTTTTCTG
TGACTGGTGAGTACTCAACCAAGTCATTCTGAGAATAGTGTATGCGGCGACCGAGTT
GCTCTTGCCCGGCGTCAATACGGGATAATACCGCGCCACATAGCAGAACTTTAAAA
GTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTA
CTTTCACCAGCGTTTCTGGGTGAGCAAAAACAGGAAGGCAAAATGCCGCAAAAAAG
GGAATAAGGGCGACACGGAAATGTTGAATACTCATACTCTTCCTTTTTCAATATTAT
TGAAGCATTTATCAGGGTTATTGTCTCATGAGCGGATACATATTTGAATGTATTTAG
AAAAATAAACAAATAGGGGTTCCGCGCACATTTCCCCGAAAAGTGCCACCTGACGT
CTAAGAAACCATTATTATCATGACATTAACCTATAAAAATAGGCGTATCACGAGGCC
CTTTCGTC
5) Protein sequence of the ORF encoded in RSV mutant F A strain mRNA
SEQ ID NO: 5
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTQATNNRARRELPRFMNYTLN
NAKKTNVTLSKKRKRRFLGFLLGVGSAIASGVAVCKVLHLEGEVNKIKSALLSTNKAVV
SLSNGVSVLTFKVLDLKNYIDKQLLPILNKQSCSISNIETVIEFQQKNNRLLEITREFSVNA
GVTTPVSTYMLTNSELLSLINDMPITNDQKKLMSNNVQIVRQQSYSIMCIIKEEVLAYVV
QLPLYGVIDTPCWKLHTSPLCTTNTKEGSNICLTRTDRGWYCDNAGSVSFFPQAETCKV
QSNRVFCDTMNSLTLPSEVNLCNVDIFNPKYDCKIMTSKTDVSSSVITSLGAIVSCYGKT
KCTASNKNRGIIKTFSNGCDYVSNKGVDTVSVGNTLYYVNKQEGKSLYVKGEPIINFYD
PLVFPSDEFDASISQVNEKINQSLAFIRKSDELLHNVNAGKSTTNIMITTIIIVIIVILLSLIAV
GLLLYCKARSTPVTLSKDQLSGINNIAFSN
6) RSV mutant F A strain mRNA sequence (ORF)
SEQ ID NO: 6
AUGGAACUGCUGAUCCUGAAGGCCAACGCUAUCACAACCAUCCUCACCGCCGUGA
CAUUCUGCUUCGCCAGCGGCCAGAACAUCACCGAAGAGUUCUACCAGAGCACCUG
CUCCGCUGUGUCUAAAGGGUACCUGUCCGCCCUGAGAACCGGCUGGUAUACCAGC
GUGAUUACGAUUGAGCUGAGCAACAUCAAGAAGAACAAGUGCAACGGAACAGAC
GCCAAGGUGAAGCUGAUCAAGCAGGAGCUGGAUAAGUAUAAGAACGCCGUGACC
GAGUUGCAGCUGCUCAUGCAGUCUACACAGGCCACAAACAAUCGGGCCAGAAGAG
AGCUGCCUAGAUUCAUGAACUACACCCUGAACAACGCCAAGAAGACAAAUGUGAC
CCUGAGCAAGAAAAGAAAGCGGAGAUUCCUGGGCUUCCUGCUCGGCGUUGGCUCU
GCCAUCGCCAGCGGAGUCGCCGUGUGCAAAGUGCUGCACCUGGAAGGAGAAGUGA
ACAAGAUCAAGAGCGCCCUGCUGUCUACGAACAAAGCCGUGGUGAGCCUGUCCAA
CGGCGUGAGCGUGCUGACCUUCAAGGUGCUGGACCUGAAGAACUACAUCGAUAA
GCAACUGCUGCCAAUCCUGAACAAGCAGUCUUGUAGCAUCAGCAAUAUCGAGACA
GUGAUCGAGUUCCAGCAGAAGAACAACCGGCUGCUGGAAAUCACACGGGAAUUU
AGCGUCAACGCCGGUGUGACCACCCCUGUGUCUACCUACAUGCUGACCAAUAGCG
AGCUGCUGAGCCUGAUCAAUGACAUGCCUAUUACCAACGACCAAAAGAAACUGAU
GAGCAACAAUGUACAAAUCGUUAGACAGCAGAGCUACUCCAUCAUGUGCAUCAUC
AAGGAGGAAGUGCUGGCCUACGUCGUGCAACUGCCUCUGUACGGCGUGAUCGACA
CCCCUUGUUGGAAGCUGCACACGAGCCCUCUGUGCACAACUAAUACAAAGGAAGG
CAGCAACAUCUGCCUGACCAGAACAGAUAGAGGCUGGUACUGCGACAACGCUGGA
UCUGUCAGCUUUUUCCCUCAGGCUGAAACCUGUAAAGUGCAGUCCAACCGCGUGU
UUUGCGAUACAAUGAACUCUCUGACACUGCCUAGCGAGGUGAACCUGUGUAAUG
UGGACAUCUUCAACCCCAAGUACGAUUGCAAGAUCAUGACAAGCAAGACCGAUGU
GUCUAGCAGCGUGAUUACCAGCCUGGGCGCCAUCGUGUCAUGCUACGGCAAGACC
AAGUGCACCGCCAGCAACAAAAAUCGGGGCAUCAUCAAAACCUUCAGCAACGGCU
GCGACUACGUGAGCAACAAAGGAGUGGACACCGUCUCCGUGGGCAACACCCUGUA
CUACGUGAACAAGCAGGAGGGCAAGUCUCUGUACGUGAAGGGCGAGCCAAUCAU
UAACUUCUACGACCCCCUGGUGUUCCCCUCUGAUGAGUUCGACGCCUCCAUCAGC
CAGGUGAACGAGAAGAUCAACCAGAGCCUGGCCUUCAUCAGAAAGAGCGACGAGC
UGCUGCACAACGUCAAUGCCGGCAAAAGCACCACCAACAUCAUGAUCACCACCAU
CAUCAUCGUGAUCAUUGUGAUCCUGCUGAGUCUGAUCGCUGUGGGCCUGCUGCUG
UAUUGUAAAGCUAGGUCCACCCCCGUGACACUUAGCAAGGACCAGCUGAGCGGCA
UCAACAACAUCGCAUUUAGCAAC
7) RSV mutant F A strain mRNA sequence (5′-UTR-ORF-3′UTR-Poly(A) tail)
SEQ ID NO: 7
AGGCCGGCACUCUUCUGGUCCCCACAGACUCAGAGAGAACCCGCCGCCACCAUGG
AACUGCUGAUCCUGAAGGCCAACGCUAUCACAACCAUCCUCACCGCCGUGACAUU
CUGCUUCGCCAGCGGCCAGAACAUCACCGAAGAGUUCUACCAGAGCACCUGCUCC
GCUGUGUCUAAAGGGUACCUGUCCGCCCUGAGAACCGGCUGGUAUACCAGCGUGA
UUACGAUUGAGCUGAGCAACAUCAAGAAGAACAAGUGCAACGGAACAGACGCCA
AGGUGAAGCUGAUCAAGCAGGAGCUGGAUAAGUAUAAGAACGCCGUGACCGAGU
UGCAGCUGCUCAUGCAGUCUACACAGGCCACAAACAAUCGGGCCAGAAGAGAGCU
GCCUAGAUUCAUGAACUACACCCUGAACAACGCCAAGAAGACAAAUGUGACCCUG
AGCAAGAAAAGAAAGCGGAGAUUCCUGGGCUUCCUGCUCGGCGUUGGCUCUGCCA
UCGCCAGCGGAGUCGCCGUGUGCAAAGUGCUGCACCUGGAAGGAGAAGUGAACA
AGAUCAAGAGCGCCCUGCUGUCUACGAACAAAGCCGUGGUGAGCCUGUCCAACGG
CGUGAGCGUGCUGACCUUCAAGGUGCUGGACCUGAAGAACUACAUCGAUAAGCA
ACUGCUGCCAAUCCUGAACAAGCAGUCUUGUAGCAUCAGCAAUAUCGAGACAGUG
AUCGAGUUCCAGCAGAAGAACAACCGGCUGCUGGAAAUCACACGGGAAUUUAGC
GUCAACGCCGGUGUGACCACCCCUGUGUCUACCUACAUGCUGACCAAUAGCGAGC
UGCUGAGCCUGAUCAAUGACAUGCCUAUUACCAACGACCAAAAGAAACUGAUGA
GCAACAAUGUACAAAUCGUUAGACAGCAGAGCUACUCCAUCAUGUGCAUCAUCAA
GGAGGAAGUGCUGGCCUACGUCGUGCAACUGCCUCUGUACGGCGUGAUCGACACC
CCUUGUUGGAAGCUGCACACGAGCCCUCUGUGCACAACUAAUACAAAGGAAGGCA
GCAACAUCUGCCUGACCAGAACAGAUAGAGGCUGGUACUGCGACAACGCUGGAUC
UGUCAGCUUUUUCCCUCAGGCUGAAACCUGUAAAGUGCAGUCCAACCGCGUGUUU
UGCGAUACAAUGAACUCUCUGACACUGCCUAGCGAGGUGAACCUGUGUAAUGUG
GACAUCUUCAACCCCAAGUACGAUUGCAAGAUCAUGACAAGCAAGACCGAUGUGU
CUAGCAGCGUGAUUACCAGCCUGGGCGCCAUCGUGUCAUGCUACGGCAAGACCAA
GUGCACCGCCAGCAACAAAAAUCGGGGCAUCAUCAAAACCUUCAGCAACGGCUGC
GACUACGUGAGCAACAAAGGAGUGGACACCGUCUCCGUGGGCAACACCCUGUACU
ACGUGAACAAGCAGGAGGGCAAGUCUCUGUACGUGAAGGGCGAGCCAAUCAUUA
ACUUCUACGACCCCCUGGUGUUCCCCUCUGAUGAGUUCGACGCCUCCAUCAGCCA
GGUGAACGAGAAGAUCAACCAGAGCCUGGCCUUCAUCAGAAAGAGCGACGAGCU
GCUGCACAACGUCAAUGCCGGCAAAAGCACCACCAACAUCAUGAUCACCACCAUC
AUCAUCGUGAUCAUUGUGAUCCUGCUGAGUCUGAUCGCUGUGGGCCUGCUGCUG
UAUUGUAAAGCUAGGUCCACCCCCGUGACACUUAGCAAGGACCAGCUGAGCGGCA
UCAACAACAUCGCAUUUAGCAACUGAUAAAGCUGGAGCCUCGGUGGCCUUGCUUC
UUGCCCCUUGGGCCUCCCCCCAGCCCCUCCUCCCCUUCCUGCACCCGUACCCCCGU
GGUCUUUGAAUAAAGUCUGAGUGGGCGGCAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAA
8) Sequence of pUC57 plasmid encoding RSV mutant F A strain mRNA (RSV
mutant F A strain mRNA sequence has been underlined)
SEQ ID NO: 8
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGGAAC
TGCTGATCCTGAAGGCCAACGCTATCACAACCATCCTCACCGCCGTGACATTCTGCT
TCGCCAGCGGCCAGAACATCACCGAAGAGTTCTACCAGAGCACCTGCTCCGCTGTGT
CTAAAGGGTACCTGTCCGCCCTGAGAACCGGCTGGTATACCAGCGTGATTACGATTG
AGCTGAGCAACATCAAGAAGAACAAGTGCAACGGAACAGACGCCAAGGTGAAGCT
GATCAAGCAGGAGCTGGATAAGTATAAGAACGCCGTGACCGAGTTGCAGCTGCTCA
TGCAGTCTACACAGGCCACAAACAATCGGGCCAGAAGAGAGCTGCCTAGATTCATG
AACTACACCCTGAACAACGCCAAGAAGACAAATGTGACCCTGAGCAAGAAAAGAA
AGCGGAGATTCCTGGGCTTCCTGCTCGGCGTTGGCTCTGCCATCGCCAGCGGAGTCG
CCGTGTGCAAAGTGCTGCACCTGGAAGGAGAAGTGAACAAGATCAAGAGCGCCCTG
CTGTCTACGAACAAAGCCGTGGTGAGCCTGTCCAACGGCGTGAGCGTGCTGACCTTC
AAGGTGCTGGACCTGAAGAACTACATCGATAAGCAACTGCTGCCAATCCTGAACAA
GCAGTCTTGTAGCATCAGCAATATCGAGACAGTGATCGAGTTCCAGCAGAAGAACA
ACCGGCTGCTGGAAATCACACGGGAATTTAGCGTCAACGCCGGTGTGACCACCCCT
GTGTCTACCTACATGCTGACCAATAGCGAGCTGCTGAGCCTGATCAATGACATGCCT
ATTACCAACGACCAAAAGAAACTGATGAGCAACAATGTACAAATCGTTAGACAGCA
GAGCTACTCCATCATGTGCATCATCAAGGAGGAAGTGCTGGCCTACGTCGTGCAACT
GCCTCTGTACGGCGTGATCGACACCCCTTGTTGGAAGCTGCACACGAGCCCTCTGTG
CACAACTAATACAAAGGAAGGCAGCAACATCTGCCTGACCAGAACAGATAGAGGCT
GGTACTGCGACAACGCTGGATCTGTCAGCTTTTTCCCTCAGGCTGAAACCTGTAAAG
TGCAGTCCAACCGCGTGTTTTGCGATACAATGAACTCTCTGACACTGCCTAGCGAGG
TGAACCTGTGTAATGTGGACATCTTCAACCCCAAGTACGATTGCAAGATCATGACAA
GCAAGACCGATGTGTCTAGCAGCGTGATTACCAGCCTGGGCGCCATCGTGTCATGCT
ACGGCAAGACCAAGTGCACCGCCAGCAACAAAAATCGGGGCATCATCAAAACCTTC
AGCAACGGCTGCGACTACGTGAGCAACAAAGGAGTGGACACCGTCTCCGTGGGCAA
CACCCTGTACTACGTGAACAAGCAGGAGGGCAAGTCTCTGTACGTGAAGGGCGAGC
CAATCATTAACTTCTACGACCCCCTGGTGTTCCCCTCTGATGAGTTCGACGCCTCCAT
CAGCCAGGTGAACGAGAAGATCAACCAGAGCCTGGCCTTCATCAGAAAGAGCGACG
AGCTGCTGCACAACGTCAATGCCGGCAAAAGCACCACCAACATCATGATCACCACC
ATCATCATCGTGATCATTGTGATCCTGCTGAGTCTGATCGCTGTGGGCCTGCTGCTGT
ATTGTAAAGCTAGGTCCACCCCCGTGACACTTAGCAAGGACCAGCTGAGCGGCATC
AACAACATCGCATTTAGCAACTGATAAAGCTGGAGCCTCGGTGGCCTTGCTTCTTGC
CCCTTGGGCCTCCCCCCAGCCCCTCCTCCCCTTCCTGCACCCGTACCCCCGTGGTCTT
TGAATAAAGTCTGAGTGGGCGGCAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAGGGCCCGTTTAAACATCGGAT
CCCGGGCCCGTCGACTGCAGAGGCCTGCATGCAAGCTTGGTGTAATCATGGTCATAG
CTGTTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAATTGC
GTTGCGCTCACTGCCCGCTTTCCAGTCGGGAAACCTGTCGTGCCAGCTGCATTAATG
AATCGGCCAACGCGCGGGGAGAGGCGGTTTGCGTATTGGGCGCTCTTCCGCTTCCTC
GCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTC
AGTGGAACGAAAACTCACGTTAAGGGATTTTGGTCATGAGATTATCAAAAAGGATC
TTCACCTAGATCCTTTTAAATTAAAAATGAAGTTTTAAATCAAGCCCAATCTGAATA
ATGTTACAACCAATTAACCAATTCTGATTAGAAAAACTCATCGAGCATCAAATGAAA
CTGCAATTTATTCATATCAGGATTATCAATACCATATTTTTGAAAAAGCCGTTTCTGT
AATGAAGGAGAAAACTCACCGAGGCAGTTCCATAGGATGGCAAGATCCTGGTATCG
GTCTGCGATTCCGACTCGTCCAACATCAATACAACCTATTAATTTCCCCTCGTCAAA
AATAAGGTTATCAAGTGAGAAATCACCATGAGTGACGACTGAATCCGGTGAGAATG
GCAAAAGTTTATGCATTTCTTTCCAGACTTGTTCAACAGGCCAGCCATTACGCTCGTC
ATCAAAATCACTCGCATCAACCAAACCGTTATTCATTCGTGATTGCGCCTGAGCGAG
ACGAAATACGCGATCGCTGTTAAAAGGACAATTACAAACAGGAATCGAATGCAACC
GGCGCAGGAACACTGCCAGCGCATCAACAATATTTTCACCTGAATCAGGATATTCTT
CTAATACCTGGAATGCTGTTTTTCCGGGGATCGCAGTGGTGAGTAACCATGCATCAT
CAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCA
GAAACAACTCTGGCGCATCGGGCTTCCCATACAAGCGATAGATTGTCGCACCTGATT
GCCCGACATTATCGCGAGCCCATTTATACCCATATAAATCAGCATCCATGTTGGAAT
TTAATCGCGGCCTCGACGTTTCCCGTTGAATATGGCTCATAACACCCCTTGTATTACT
GTTTATGTAAGCAGACAGTTTTATTGTTCATGATGATATATTTTTATCTTGTGCAATG
TAACATCAGAGATTTTGAGACACGGGCCAGAGCTGCA
9) Protein sequence of the ORF encoded in hMPV-F mRNA
SEQ ID NO: 9
MSWKVVIIFSLLITPQHGLKESYLEESCSTITEGYLSVLRTGWYTNVFTLEVGDVENLTC
ADGPSLIKTELDLTKSALRELKTVSADQLAREEQIENPRQSRFVLGAIALGVATAAAVTA
GVAIAKTIRLESEVTAIKNALKKTNEAVSTLGNGVRVLATAVRELKDFVSKNLTRAINK
NKCDIDDLKMAVSFSQFNRRFLNVVRQFSDNAGITPAISLDLMTDAELARAVSNMPTSA
GQIKLMLENRAMVRRKGFGILIGVYGSSVIYMVOLPIFGVIDTPCWIVKAAPSCSEKKGN
YACLLREDQGWYCQNAGSTVYYPNEKDCETRGDHVFCDTAAGINVAEQSKECNINIST
TNYPCKVSTGRHPISMVALSPLGALVACYKGVSCSIGSNRVGIIKQLNKGCSYITNQDAD
TVTIDNTVYQLSKVEGEQHVIKGRPVSSSFDPVKFPEDQFNVALDQVFENIENSQALVDQ
SNRILSSAEKGNTGFIIVIILIAVLGSSMILVSICIIIKKTKKPTGAPPELSGVTNNGFIPHS
10) hMPV-F mRNA (ORF)
SEQ ID NO: 10
AUGAGCUGGAAGGUGGUGAUCAUCUUCAGCCUGCUGAUCACCCCUCAGCACGGCC
UGAAAGAGAGCUACCUGGAAGAAAGCUGCUCUACCAUCACAGAGGGCUACCUGA
GUGUGCUGCGGACCGGCUGGUACACAAACGUGUUUACCCUGGAAGUGGGCGACG
UGGAAAACCUGACAUGCGCCGAUGGCCCCAGCCUGAUUAAGACAGAGCUGGACCU
GACAAAGAGCGCCCUGAGAGAACUGAAGACAGUCAGCGCUGAUCAACUGGCCAGG
GAGGAGCAGAUCGAGAACCCCAGACAGUCCAGAUUCGUGCUCGGUGCCAUCGCCC
UGGGCGUGGCGACAGCUGCCGCUGUUACCGCUGGCGUGGCUAUCGCCAAGACCAU
CAGACUGGAAUCUGAGGUGACCGCCAUCAAGAAUGCCCUGAAGAAGACCAAUGA
GGCCGUGUCUACACUGGGAAAUGGCGUGCGGGUGCUGGCUACAGCCGUCCGGGAA
CUGAAAGACUUCGUGUCCAAGAACUUGACCAGAGCCAUCAACAAGAACAAGUGCG
AUAUCGACGACCUGAAGAUGGCCGUGAGCUUCAGCCAGUUCAACCGGCGGUUCCU
GAACGUUGUGCGGCAGUUUAGCGACAACGCCGGAAUCACCCCAGCCAUCAGCCUG
GACCUGAUGACCGACGCCGAGCUGGCCAGAGCCGUGAGCAACAUGCCUACAAGCG
CCGGCCAGAUCAAGCUGAUGCUGGAGAAUCGGGCCAUGGUGAGAAGAAAGGGCU
UUGGCAUCCUGAUCGGCGUGUACGGCAGCAGCGUGAUCUACAUGGUGCAGCUGCC
UAUCUUCGGCGUGAUCGAUACCCCUUGCUGGAUCGUGAAGGCCGCCCCUAGCUGU
AGCGAGAAAAAGGGAAACUACGCCUGUCUGCUGAGAGAAGAUCAGGGCUGGUAU
UGCCAGAACGCCGGCAGCACCGUGUACUACCCCAACGAGAAGGACUGCGAGACAA
GAGGCGAUCACGUGUUCUGCGACACCGCCGCCGGCAUCAACGUUGCUGAGCAAAG
CAAGGAAUGCAACAUCAACAUCAGCACCACCAACUACCCUUGUAAAGUGAGCACC
GGAAGACACCCCAUCUCCAUGGUCGCACUCUCCCCUCUGGGCGCCCUGGUCGCCU
GCUACAAGGGCGUGUCUUGUAGCAUCGGAAGCAACCGCGUUGGCAUCAUCAAGCA
GCUGAACAAAGGAUGUUCUUAUAUCACCAACCAGGAUGCCGACACCGUGACAAUC
GACAAUACCGUCUACCAGCUGAGCAAGGUGGAAGGCGAGCAGCACGUGAUUAAG
GGCAGACCUGUGUCCUCUAGCUUCGACCCCGUGAAGUUCCCCGAGGACCAGUUCA
AUGUGGCCCUGGAUCAAGUGUUUGAGAACAUCGAGAACAGCCAGGCCCUGGUGG
ACCAGAGCAAUAGAAUCCUGUCCUCCGCUGAGAAAGGCAACACCGGCUUCAUCAU
CGUGAUCAUCCUGAUCGCCGUGCUGGGCUCUAGCAUGAUCCUGGUGUCUAUCUGC
AUUAUUAUCAAGAAAACCAAGAAGCCUACCGGCGCUCCACCUGAGCUGAGCGGAG
UGACCAACAACGGCUUCAUCCCUCAUUCU
11) hMPV-F mRNA sequence (5′-UTR-ORF-3′UTR-Poly(A) tail)
SEQ ID NO: 11
AGGCCGGCACUCUUCUGGUCCCCACAGACUCAGAGAGAACCCGCCGCCACCAUGA
GCUGGAAGGUGGUGAUCAUCUUCAGCCUGCUGAUCACCCCUCAGCACGGCCUGAA
AGAGAGCUACCUGGAAGAAAGCUGCUCUACCAUCACAGAGGGCUACCUGAGUGU
GCUGCGGACCGGCUGGUACACAAACGUGUUUACCCUGGAAGUGGGCGACGUGGA
AAACCUGACAUGCGCCGAUGGCCCCAGCCUGAUUAAGACAGAGCUGGACCUGACA
AAGAGCGCCCUGAGAGAACUGAAGACAGUCAGCGCUGAUCAACUGGCCAGGGAG
GAGCAGAUCGAGAACCCCAGACAGUCCAGAUUCGUGCUCGGUGCCAUCGCCCUGG
GCGUGGCGACAGCUGCCGCUGUUACCGCUGGCGUGGCUAUCGCCAAGACCAUCAG
ACUGGAAUCUGAGGUGACCGCCAUCAAGAAUGCCCUGAAGAAGACCAAUGAGGCC
GUGUCUACACUGGGAAAUGGCGUGCGGGUGCUGGCUACAGCCGUCCGGGAACUG
AAAGACUUCGUGUCCAAGAACUUGACCAGAGCCAUCAACAAGAACAAGUGCGAU
AUCGACGACCUGAAGAUGGCCGUGAGCUUCAGCCAGUUCAACCGGCGGUUCCUGA
ACGUUGUGCGGCAGUUUAGCGACAACGCCGGAAUCACCCCAGCCAUCAGCCUGGA
CCUGAUGACCGACGCCGAGCUGGCCAGAGCCGUGAGCAACAUGCCUACAAGCGCC
GGCCAGAUCAAGCUGAUGCUGGAGAAUCGGGCCAUGGUGAGAAGAAAGGGCUUU
GGCAUCCUGAUCGGCGUGUACGGCAGCAGCGUGAUCUACAUGGUGCAGCUGCCUA
UCUUCGGCGUGAUCGAUACCCCUUGCUGGAUCGUGAAGGCCGCCCCUAGCUGUAG
CGAGAAAAAGGGAAACUACGCCUGUCUGCUGAGAGAAGAUCAGGGCUGGUAUUG
CCAGAACGCCGGCAGCACCGUGUACUACCCCAACGAGAAGGACUGCGAGACAAGA
GGCGAUCACGUGUUCUGCGACACCGCCGCCGGCAUCAACGUUGCUGAGCAAAGCA
AGGAAUGCAACAUCAACAUCAGCACCACCAACUACCCUUGUAAAGUGAGCACCGG
AAGACACCCCAUCUCCAUGGUCGCACUCUCCCCUCUGGGCGCCCUGGUCGCCUGC
UACAAGGGCGUGUCUUGUAGCAUCGGAAGCAACCGCGUUGGCAUCAUCAAGCAGC
UGAACAAAGGAUGUUCUUAUAUCACCAACCAGGAUGCCGACACCGUGACAAUCGA
CAAUACCGUCUACCAGCUGAGCAAGGUGGAAGGCGAGCAGCACGUGAUUAAGGG
CAGACCUGUGUCCUCUAGCUUCGACCCCGUGAAGUUCCCCGAGGACCAGUUCAAU
GUGGCCCUGGAUCAAGUGUUUGAGAACAUCGAGAACAGCCAGGCCCUGGUGGACC
AGAGCAAUAGAAUCCUGUCCUCCGCUGAGAAAGGCAACACCGGCUUCAUCAUCGU
GAUCAUCCUGAUCGCCGUGCUGGGCUCUAGCAUGAUCCUGGUGUCUAUCUGCAUU
AUUAUCAAGAAAACCAAGAAGCCUACCGGCGCUCCACCUGAGCUGAGCGGAGUGA
CCAACAACGGCUUCAUCCCUCAUUCUUGACUCGAGUAAGCUGGAGCCUCGGUGGC
CAUGCUUCUUGCCCCUUGGGCCUCCCCCCAGCCCCUCCUCCCCUUCCUGCACCCGU
ACCCCCGUGGUCUUUGAAUAAAGUCUGAGUGGGCGGCAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAA
12) Sequence of pUC57 plasmid encoding hMPV-F mRNA (hMPV-F mRNA sequence
has been underlined)
SEQ ID NO: 12
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGAGCT
GGAAGGTGGTGATCATCTTCAGCCTGCTGATCACCCCTCAGCACGGCCTGAAAGAG
AGCTACCTGGAAGAAAGCTGCTCTACCATCACAGAGGGCTACCTGAGTGTGCTGCG
GACCGGCTGGTACACAAACGTGTTTACCCTGGAAGTGGGCGACGTGGAAAACCTGA
CATGCGCCGATGGCCCCAGCCTGATTAAGACAGAGCTGGACCTGACAAAGAGCGCC
CTGAGAGAACTGAAGACAGTCAGCGCTGATCAACTGGCCAGGGAGGAGCAGATCGA
GAACCCCAGACAGTCCAGATTCGTGCTCGGTGCCATCGCCCTGGGCGTGGCGACAG
CTGCCGCTGTTACCGCTGGCGTGGCTATCGCCAAGACCATCAGACTGGAATCTGAGG
TGACCGCCATCAAGAATGCCCTGAAGAAGACCAATGAGGCCGTGTCTACACTGGGA
AATGGCGTGCGGGTGCTGGCTACAGCCGTCCGGGAACTGAAAGACTTCGTGTCCAA
GAACTTGACCAGAGCCATCAACAAGAACAAGTGCGATATCGACGACCTGAAGATGG
CCGTGAGCTTCAGCCAGTTCAACCGGCGGTTCCTGAACGTTGTGCGGCAGTTTAGCG
ACAACGCCGGAATCACCCCAGCCATCAGCCTGGACCTGATGACCGACGCCGAGCTG
GCCAGAGCCGTGAGCAACATGCCTACAAGCGCCGGCCAGATCAAGCTGATGCTGGA
GAATCGGGCCATGGTGAGAAGAAAGGGCTTTGGCATCCTGATCGGCGTGTACGGCA
GCAGCGTGATCTACATGGTGCAGCTGCCTATCTTCGGCGTGATCGATACCCCTTGCT
GGATCGTGAAGGCCGCCCCTAGCTGTAGCGAGAAAAAGGGAAACTACGCCTGTCTG
CTGAGAGAAGATCAGGGCTGGTATTGCCAGAACGCCGGCAGCACCGTGTACTACCC
CAACGAGAAGGACTGCGAGACAAGAGGCGATCACGTGTTCTGCGACACCGCCGCCG
GCATCAACGTTGCTGAGCAAAGCAAGGAATGCAACATCAACATCAGCACCACCAAC
TACCCTTGTAAAGTGAGCACCGGAAGACACCCCATCTCCATGGTCGCACTCTCCCCT
CTGGGCGCCCTGGTCGCCTGCTACAAGGGCGTGTCTTGTAGCATCGGAAGCAACCGC
GTTGGCATCATCAAGCAGCTGAACAAAGGATGTTCTTATATCACCAACCAGGATGCC
GACACCGTGACAATCGACAATACCGTCTACCAGCTGAGCAAGGTGGAAGGCGAGCA
GCACGTGATTAAGGGCAGACCTGTGTCCTCTAGCTTCGACCCCGTGAAGTTCCCCGA
GGACCAGTTCAATGTGGCCCTGGATCAAGTGTTTGAGAACATCGAGAACAGCCAGG
CCCTGGTGGACCAGAGCAATAGAATCCTGTCCTCCGCTGAGAAAGGCAACACCGGC
TTCATCATCGTGATCATCCTGATCGCCGTGCTGGGCTCTAGCATGATCCTGGTGTCTA
TCTGCATTATTATCAAGAAAACCAAGAAGCCTACCGGCGCTCCACCTGAGCTGAGC
GGAGTGACCAACAACGGCTTCATCCCTCATTCTTGACTCGAGTAAGCTGGAGCCTCG
GTGGCCATGCTTCTTGCCCCTTGGGCCTCCCCCCAGCCCCTCCTCCCCTTCCTGCACC
CGTACCCCCGTGGTCTTTGAATAAAGTCTGAGTGGGCGGCAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAATGAA
GAGCATCGGATCCCGGGCCCGTCGACTGCAGAGGCCTGCATGCAAGCTTGGTGTAA
TCATGGTCATAGCTGTTTCCTGTGTGAAATTGTTATCCGCTCACAATTCCACACAACA
TACGAGCCGGAAGCATAAAGTGTAAAGCCTGGGGTGCCTAATGAGTGAGCTAACTC
ACATTAATTGCGTTGCGCTCACTGCCCGCTTTCCAGTCGGGAAACCTGTCGTGCCAG
CTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGG
AAAGAACATGTGAGCAAAAGGCCAGCAAAAGGCCAGGAACCGTAAAAAGGCCGCG
TTGCTGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCCCCC
TGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGAAGAAC
AGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAAGCCC
AATCTGAATAATGTTACAACCAATTAACCAATTCTGATTAGAAAAACTCATCGAGCA
TCAAATGAAACTGCAATTTATTCATATCAGGATTATCAATACCATATTTTTGAAAAA
GCCGTTTCTGTAATGAAGGAGAAAACTCACCGAGGCAGTTCCATAGGATGGCAAGA
TCCTGGTATCGGTCTGCGATTCCGACTCGTCCAACATCAATACAACCTATTAATTTCC
CCTCGTCAAAAATAAGGTTATCAAGTGAGAAATCACCATGAGTGACGACTGAATCC
GGTGAGAATGGCAAAAGTTTATGCATTTCTTTCCAGACTTGTTCAACAGGCCAGCCA
TTACGCTCGTCATCAAAATCACTCGCATCAACCAAACCGTTATTCATTCGTGATTGC
GCCTGAGCGAGACGAAATACGCGATCGCTGTTAAAAGGACAATTACAAACAGGAAT
CGAATGCAACCGGCGCAGGAACACTGCCAGCGCATCAACAATATTTTCACCTGAAT
CAGGATATTCTTCTAATACCTGGAATGCTGTTTTTCCGGGGATCGCAGTGGTGAGTA
ACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTT
TGCCATGTTTCAGAAACAACTCTGGCGCATCGGGCTTCCCATACAAGCGATAGATTG
TCGCACCTGATTGCCCGACATTATCGCGAGCCCATTTATACCCATATAAATCAGCAT
CCATGTTGGAATTTAATCGCGGCCTCGACGTTTCCCGTTGAATATGGCTCATAACAC
CCCTTGTATTACTGTTTATGTAAGCAGACAGTTTTATTGTTCATGATGATATATTTTT
ATCTTGTGCAATGTAACATCAGAGATTTTGAGACACGGGCCAGAGCTGCA

### EXAMPLES

### Example 1—DNA Templates Used for In Vitro Transcription and Protein Expression of RSV F Genes

DNA constructs used for RSV mutant F B strain mRNA productions (in vitro transcription, IVT) consist of 1) T7 promoter, 2) 5′ untranslated region (UTR), 3) open reading frame (ORF) of (RSV mutant F B strain) modified from human RSV strain B membrane glycoprotein DNA (GenBank: MZ515553.1), 4) 3′UTR, and 5) 120 bases of poly adenine (polyA). 5′UTR and 3′UTR sequences were from human hemoglobin subunit alpha 1 (HBA1) mRNA (GenBank: NM_000558.5). The sequence of pUC57 plasmid encoding RSV mutant F B strain mRNA is shown in SEQ ID NO: 4.

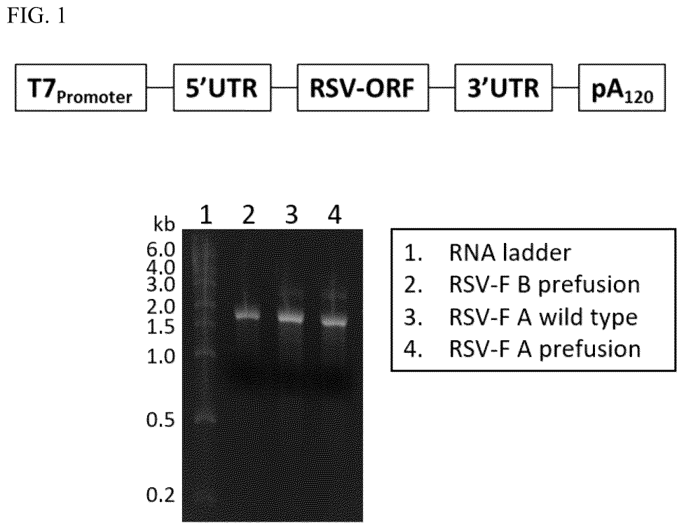

The DNA fragment was synthesized and subcloned into pUC57-Kan vector by GenScript (Piscataway, NJ: https://www.genscript.com/vector/SD1176-pUC57_plasmid_DNA.html). For a stabilized prefusion form of RSV-F protein, a mutant RSV-F (Fmut) with four amino acid substitutions (S155C, S190F, V207L, and S290C), was generated from a mutagenesis on the RSV-F clone by GenScript (FIG. 1 top).

DNA construct used for RSV mutant F A strain mRNA productions (in vitro transcription, IVT) consist of 1) T7 promoter, 2) 5′ untranslated region (UTR), 3) open reading frame (ORF) of RSV mutant F A strain modified from human RSV strain A2 membrane glycoprotein DNA (GenBank: KT992094.1), 4) 3′UTR, and 5) 120 bases of poly adenine (polyA). 5′UTR and 3′UTR sequences are from human hemoglobin subunit alpha 1 (HBA1) mRNA (GenBank: NM_000558.5). The sequence of pUC57 plasmid encoding RSV mutant F A strain is shown in SEQ ID NO: 8.

The DNA fragment was synthesized and subcloned into pUC57-Kan vector by GenScript (Piscataway, NJ: https://www.genscript.com/vector/SD1176-pUC57_plasmid_DNA.html). For a stabilized prefusion form of RSV-F protein, a mutant RSV-F (Fmut) with four amino acid substitutions (S155C, S190F, V207L, and S290C), was generated from a mutagenesis on the RSV-F clone by GenScript.

### Example 2—In Vitro Transcription (IVT)

The plasmid vector was linearized by restriction enzyme, PmeI or SapI (New England Biolabs) for RSV F IVTs. N1-Methylpseudouridine (m1Ψ) was purchased from BOC Sciences (Shirley, NY). IVT condition was followed by manufacture's recommendation (TranscriptAid T7 High Yield Transcription Kit, ThermoFisher) as below:
ATP/CTP/GTP/m1ψTP: 5 mM each
SmartCap (SC101, ST Pharm): 4 mM
Linear template DNA: 1 ug of plasmid or 0.5 ug of PCR product
T7 RNA polymerase enzyme mix: 2 ul

IVT was carried out in 20 ul reaction incubated at 37° ° C. for 2 hours. The template DNA was removed by 2 units of DNase I (ThermoFisher) to be treated at 37° C. for 15 min followed by a column purification (Monarch RNA Cleanup Kit, New England Biolabs).

The IVT product of RSV mutant F B strain gene and the IVT product of RSV mutant F A strain gene were analyzed by agarose gel, and their 2knt long mRNA products were detected. Here, after IVTs from DNA templates of RSV mutant F B strain and RSV mutant F A strain mRNAs, 100 ng of mRNA was run on 1% agarose of E-GEL EX in E-Gel Power Snap Electrophoresis Device (ThermoFisher) shown in FIG. 1 bottom.

### Example 3—Transfection and Western Blots

1 ug of each mRNAs were transfected into 293FT cells or SJCRH30 (CRL-2061, ATCC) in 12 well plate using Lipofectamine MesseangerMax, 2 or 4 ul at 1:2 ratio (ThermoFisher) according to the manufacturer's protocol. Cell lysate was prepared in NP-40 lysis buffer (150 mM sodium chloride/1% NP-40/50 mM Tris pH8.0) after 24 hours of transfection.

Mouse monoclonal anti-RSV-F antibody (NB110-37246, Noivus Biologicals) was used for Western Blot. Detection of protein was using a HRP-conjugated goat anti-mouse IgG antibody (115-035-062, Jackson ImmunoResearch) and SuperSignal West Pico Plus Chemiluminescent Substrate (ThermoScientific).

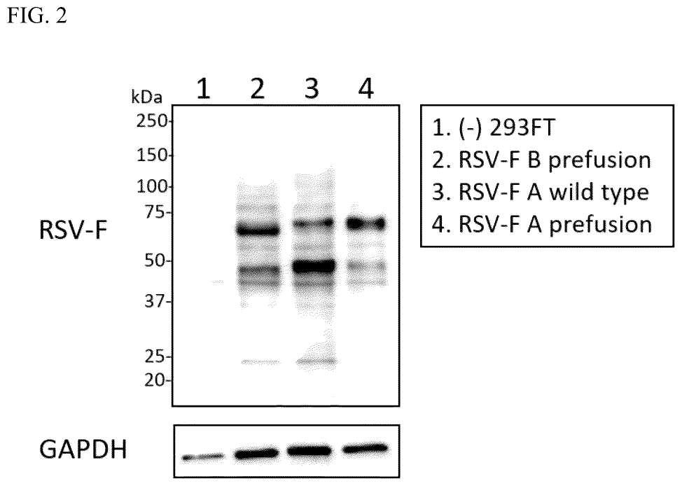

The expression of RSV mutant F proteins were detected in the transfected 293 FT cells with individual mRNAs (FIG. 2). Here, 1 ug RSV mRNAs were individually transfected into 293FT cells. The cell lysates were subjected to Western Blot, and (RSV mutant F from B and A strains)(lane 2 and 4) were detected as shown in FIG. 2. Lanes 1: negative control.

### Example 4—Immunogenicity Study for RSV Vaccine Composition

This study was designed to test the immunogenicity in mice of the RSV vaccine composition of the present disclosure.

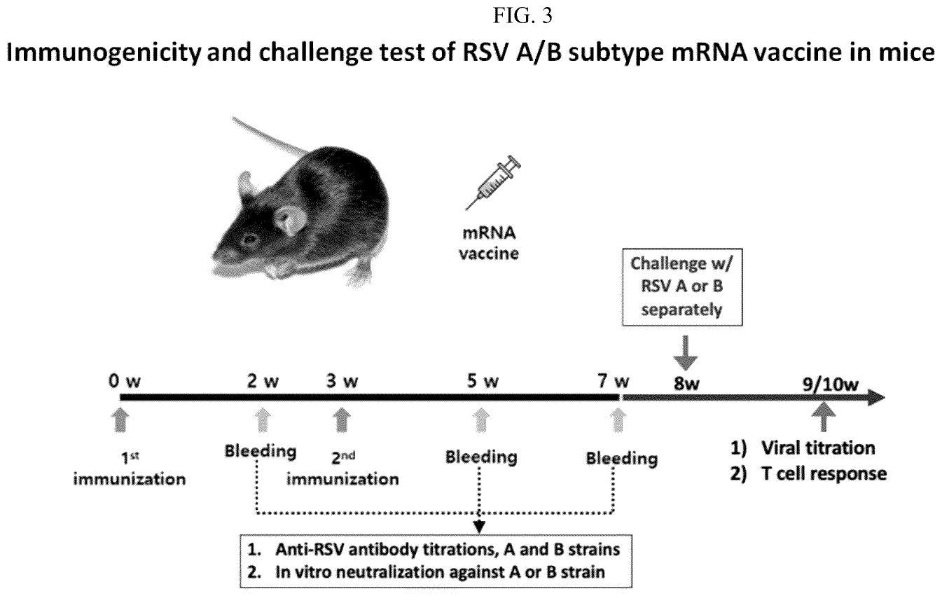

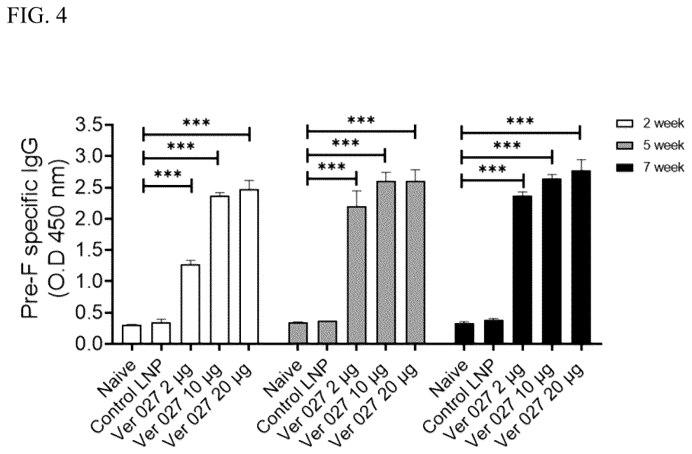

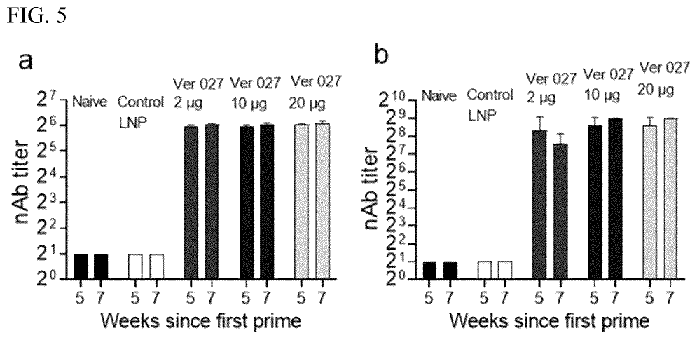

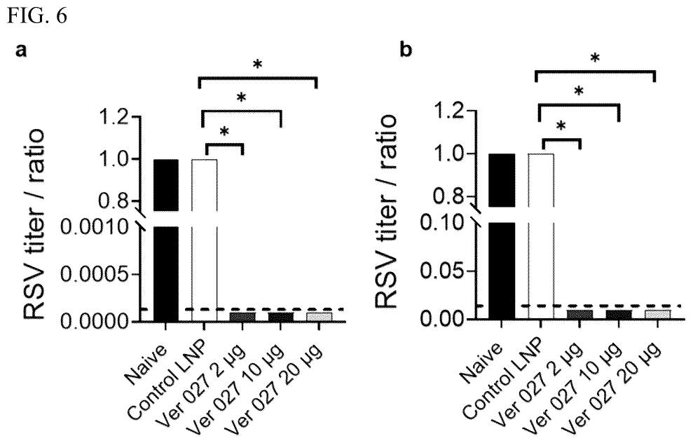

Mice were immunized intramuscularly (IM) with the RSV vaccine composition of the present disclosure (RSV mutant F B strain, and RSV mutant F A strain). The vaccine composition of the present disclosure was chemically modified or unmodified. A total of two immunizations are given at 3-week intervals (i.e., at weeks 0 and 3), and sera were collected after each immunization until weeks 7 (weeks 2, 5 and 7) (FIG. 3). Serum IgG titers specific to Pre-F protein in the sera collected at weeks 2, 5, and 7, were determined by ELISA (FIG. 4). The neutralization titers of the pooled sera from weeks 2, 5 and 7 against RSV A and B strains were separately determined by plaque assay-based neutralization assay (FIG. 5). To detect the T cell response of RSV vaccine in the vaccinated mice, the spleens in the immunized and RSV challenged (4 days) mice were harvested and stained with RSV F85-93 tetramer (FIG. 6). % of CD8+ T cells stained with F85-93 tetramer were determined by flow cytometric analysis (FIG. 6).

### Example 5—Virus Challenge Study for RSV Vaccine Composition

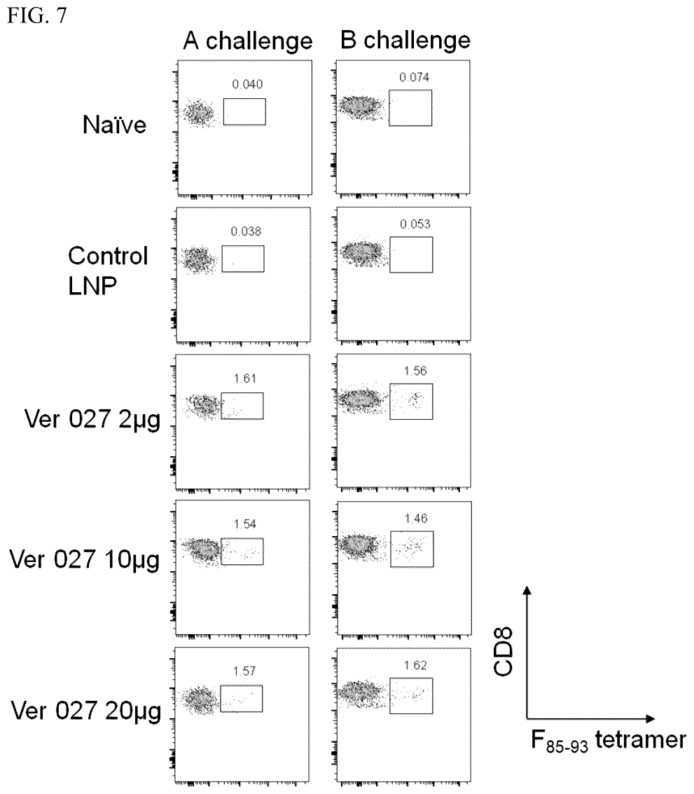

This study was designed to evaluate the protection efficacy of the RSV vaccine against RSV subtype A and subtype B infection in mice post immunization with the proper doses and formulations described in the Immunogenicity study. The mice immunized twice at weeks of 0 and 3 were divided into two groups, and each group was separately infected by RSV A2 strain or RSV B1 strain at week 8. In vivo lung viral titers were monitored at days 4 post infection. Viral titers isolated from the collected lungs were determined by plaque assay for RSV A2 and Q-RT-PCR for RSV B1 (FIG. 7).

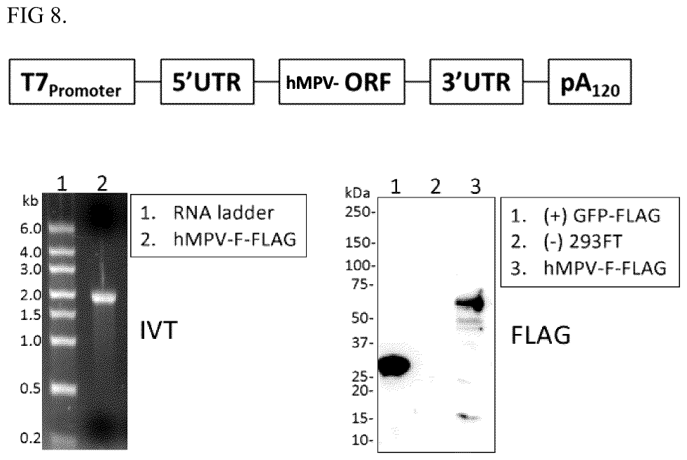

### Example 6—DNA Templates Used for In Vitro Transcription and Protein Expression of hMPV F Genes (See FIG. 8)

DNA constructs used for hPMV F protein mRNA was prepared as shown in FIG. 8 Top.

IVT (FIG. 8 Bottom left): The hMPV F protein mRNA was produced as described at FIG. 8 Bottom Left.

Western blot (FIG. 8 Bottom right): The detection of hMPV F protein from mRNA was conducted as described above for the RSV F proten. Mouse monoclonal anti-FLAG antibody (AB_1957945, Invitrogen) was used for FLAG-tagged hMPV-F protein. The expression of hMPV F protein was detected in the transfected 293 FT cells with mRNA having a FLAG tag at 3′ end of hMPV-F ORF as shown in FIG. 8.

### Example 7—Immunogenicity Study for RSV Vaccine Composition and hMPV Vaccine Composition

This study is designed to test the immunogenicity in mice of the RSV vaccine composition or the RSV and hMPV vaccine composition of the present disclosure.

Mice are immunized intramuscularly (IM) or intradermally (ID) with the RSV vaccine composition of the present disclosure (RSV mutant F B strain, and RSV mutant F A strain) or the RSV and hMPV vaccine composition of the present disclosure (RSV mutant F B strain, RSV mutant F A strain, and hMPV F). The vaccine composition of the present disclosure is chemically modified or unmodified. A total of four immunizations are given at 3-week intervals (i.e., at weeks 0 and 3), and sera are collected after each immunization until weeks 8. Serum antibody titers against RSV mutant F B strain, RSV mutant F A strain, and hMPV F are determined by ELISA. Sera collected from each mouse during weeks 10-16 are, optionally, pooled, and used for virus neutralization assay. Purified IgG antibodies are used for immunoelectron microscopy, antibody-affinity testing, and in vitro protection assays. The spleen in the immunized mice will be harvested and used for T cell responses for various cytokines including TNF alpha, INF-gamma, and IL-2 post peptide stimulation. Doses and formulations will be selected for the virus challenge study.

### Example 8—Virus Challenge Study for RSV Vaccine Composition and hMPV Vaccine Composition

This study is designed to evaluate the protection efficacy of the RSV vaccine or the RSV and hMPV vaccine against RSV infection and/or hMPV infection in mice post immunization with the proper doses and formulations determined in the Immunogenicity study. The mice will be immunized twice at weeks of 0 and 3, and the immunized mice will be infected by RSV and/or hMPV at week 6. Phenotypes (weight loss, fever, and survival) will be monitored for 2 weeks post infection. Viral titers in sera collected at weeks 1 and 2 post infection will be determined. The sacrificed mice will be examined for pathology.

1. A respiratory syncytial virus (RSV) vaccine composition comprising a messenger ribonucleic acid (mRNA) comprising an open reading frame (ORF) encoding RSV mutant F B strain protein having an amino acid sequence of SEQ ID NO: 1.

2. The RSV vaccine composition according to claim 1, wherein the ORF encoding RSV mutant F B strain protein has a nucleotide sequence of SEQ ID NO: 2.

3. The RSV vaccine composition according to claim 2, wherein the mRNA comprising the ORF encoding RSV mutant F B strain protein further comprises a 5′ untranslated region (UTR), a 3′ UTR, and a poly (A) tail so as to have the following structure:
5′UTR-ORF encoding RSV mutant F B strain protein-3′UTR-poly (A) tail, and
wherein the ORF encoding RSV mutant F B strain protein has a nucleotide sequence of SEQ ID NO: 2.

4. The RSV vaccine composition according to claim 3, wherein the poly (A) tail has a length of 50-250 nucleotides.

5. The RSV vaccine composition according to claim 3, wherein the mRNA having the structure of 5′UTR-ORF encoding RSV mutant F B strain protein-3′UTR-poly (A) tail has a nucleotide sequence of SEQ ID NO: 3.

6. The RSV vaccine composition according to claim 3, wherein the mRNA having the structure of 5′UTR-ORF encoding RSV mutant F B strain protein-3′UTR-poly (A) tail has a nucleotide sequence having at least 80% identity to SEQ ID NO: 3.

7. The RSV vaccine composition according to claim 1 further comprising a pharmaceutically acceptable carrier.

8. The RSV vaccine composition according to claim 7, wherein the pharmaceutically acceptable carrier is a lipid nanoparticle encapsulating the mRNA therein.

9. A method of inducing immune response against respiratory syncytial virus (RSV) comprising:
administering an effective amount of the RSV vaccine composition according to claim 1 to a subject in need thereof.

10. The RSV vaccine composition according to claim 1 further comprising a mRNA comprising an ORF encoding RSV mutant F A strain protein having an amino acid sequence of SEQ ID NO: 5.

11. The RSV vaccine composition according to claim 10, wherein the ORF encoding RSV mutant F A strain protein has a nucleotide sequence of SEQ ID NO: 6.

12. The RSV vaccine composition according to claim 11, wherein the mRNA comprising the ORF encoding RSV mutant F A strain protein further comprises a 5′ untranslated region (UTR), a 3′ UTR, and a poly (A) tail so as to have the following structure:
5′UTR-ORF encoding RSV mutant F A strain protein-3′UTR-poly (A) tail, and
wherein the ORF encoding RSV mutant F A strain protein has a nucleotide sequence of SEQ ID NO: 6.

13. The RSV vaccine composition according to claim 12, wherein the poly (A) tail has a length of 50-250 nucleotides.

14. The RSV vaccine composition according to claim 12, wherein the mRNA having the structure of 5′UTR-ORF encoding RSV mutant F A strain protein-3′UTR-poly (A) tail has a nucleotide sequence of SEQ ID NO: 7.

15. The RSV vaccine composition according to claim 12, wherein the mRNA having the structure of 5′UTR-ORF encoding RSV mutant F A strain protein-3′UTR-poly (A) tail has a nucleotide sequence having at least 80% identity to SEQ ID NO: 7.

16. The RSV vaccine composition according to claim 10 further comprising a pharmaceutically acceptable carrier.

17. The RSV vaccine composition according to claim 16, wherein the pharmaceutically acceptable carrier is a lipid nanoparticle encapsulating the mRNA therein.

18. A method of inducing immune response against respiratory syncytial virus (RSV) comprising:
administering an effective amount of the RSV vaccine composition according to claim 10 to a subject in need thereof.

19. A respiratory syncytial virus (RSV) and human metapneumovirus virus (hMPV) vaccine composition comprising
a messenger ribonucleic acid (mRNA) comprising an open reading frame (ORF) encoding RSV mutant F A strain protein having an amino acid sequence of SEQ ID NO: 5,
a mRNA comprising an ORF encoding RSV mutant F B strain protein having an amino acid sequence of SEQ ID NO: 1, and
a mRNA comprising an ORF encoding hMPV F protein having an amino acid sequence of SEQ ID NO: 9.

20. The RSV and hMPV vaccine composition according to claim 19,
wherein the ORF encoding RSV mutant F A strain protein has a nucleotide sequence of SEQ ID NO: 6,
wherein the ORF encoding RSV mutant F B strain protein has a nucleotide sequence of SEQ ID NO: 2, and
wherein the ORF encoding hMPV F protein has a nucleotide sequence of SEQ ID NO: 10.

21. The RSV and hMPV vaccine composition according to claim 20,
(i) wherein the mRNA comprising the ORF encoding RSV mutant F A strain protein further comprises a 5′ untranslated region (UTR), a 3′ UTR, and a poly (A) tail so as to have the following structure:
5′UTR-ORF encoding RSV mutant F A strain protein-3′UTR-poly (A) tail, and
wherein the ORF encoding RSV mutant F A strain protein has a nucleotide sequence of SEQ ID NO: 6,
(ii) wherein the mRNA comprising the ORF encoding RSV mutant F B strain protein further comprises a 5′ untranslated region (UTR), a 3′ UTR, and a poly (A) tail so as to have the following structure:
5′UTR-ORF encoding RSV mutant F B strain protein-3′UTR-poly (A) tail, and
wherein the ORF encoding RSV mutant F B strain protein has a nucleotide sequence of SEQ ID NO: 2, and
(iii) wherein the mRNA comprising the ORF encoding hMPV F protein further comprises a 5′ untranslated region (UTR), a 3′ UTR, and a poly (A) tail so as to have the following structure:
5′UTR-ORF encoding hMPV F protein-3′UTR-poly (A) tail, and
wherein the ORF encoding hMPV F protein has a nucleotide sequence of SEQ ID NO: 10.

22. The RSV and hMPV vaccine composition according to claim 21, wherein the poly (A) tail has a length of 50-250 nucleotides.

23. The RSV and hMPV vaccine composition according to claim 21,
(i) wherein the mRNA having the structure of 5′UTR-ORF encoding RSV mutant F A strain protein-3′UTR-poly (A) tail has a nucleotide sequence of SEQ ID NO: 7,
(ii) wherein the mRNA having the structure of 5′UTR-ORF encoding RSV mutant F B strain protein-3′UTR-poly (A) tail has a nucleotide sequence of SEQ ID NO: 3, and
(iii) wherein the mRNA having the structure of 5′UTR-ORF encoding hMPV F protein-3′UTR-poly (A) tail has a nucleotide sequence of SEQ ID NO: 11.

24. The RSV and hMPV vaccine composition according to claim 21,
(i) wherein the mRNA having the structure of 5′UTR-ORF encoding RSV mutant F A strain protein-3′UTR-poly (A) tail has a nucleotide sequence having at least 80% identity to SEQ ID NO: 7,
(ii) wherein the mRNA having the structure of 5′UTR-ORF encoding RSV mutant F B strain protein-3′UTR-poly (A) tail has a nucleotide sequence having at least 80% identity to SEQ ID NO: 3, and
(iii) wherein the mRNA having the structure of 5′UTR-ORF encoding hMPV F protein-3′UTR-poly (A) tail has a nucleotide sequence having at least 80% identity to SEQ ID NO: 11.

25. The RSV and hMPV vaccine composition according to claim 19 further comprising a pharmaceutically acceptable carrier.

26. The RSV and hMPV vaccine composition according to claim 25, wherein the pharmaceutically acceptable carrier is a lipid nanoparticle encapsulating the mRNA therein.

27. A method of inducing immune response against respiratory syncytial virus (RSV) and human metapneumovirus virus (hMPV) comprising:
administering an effective amount of the RSV and hMPV vaccine composition according to claim 19 to a subject in need thereof.
